(19) United States
(12) Patent Application Publication
(10) Pub. No.: US 2006/0183902 A1
(43) Pub. Date: Aug. 17, 2006

(54) Dihydroindolyl methanones as alpha 1a/1d adrenoreceptor modulators for the treatment of benign prostatic hypertrophy and lower urinary tract symptoms

(76) Inventors: Ellen W. Baxter, Glenside, PA (US); Samuel O. Nortey, Elkins Park, PA (US); Allen B. Reitz, Lansdale, PA (US); Virginia L. Pulito, Flemington, NJ (US); Steven A. Middleton, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/353,581

(22) Filed: Feb. 14, 2006

Related U.S. Application Data
(60) Provisional application No. 60/653,218, filed on Feb. 15, 2005.

(51) Int. Cl. C07D 401/00; A61K 31/497; C07D 403/00

(52) U.S. Cl. 544/373; 514/254.09; 514/323; 546/201

(57) ABSTRACT

The present invention relates to new compounds of Formula (I): 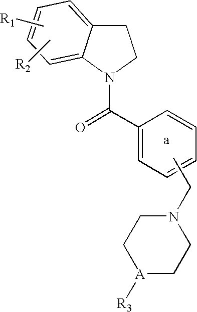
and pharmaceutically acceptable forms thereof, use of the compounds as α₁ₐ and/or α_{1d} adrenoreceptor modulators, including use of a pharmaceutical composition, medicine or medicament comprising said compounds, a process to prepare said compounds and a method for treating an α₁ₐ and/or α_{1d} adrenoreceptor mediated disorder.

### CROSS REFERENCE TO RELATED APPLICATIONS

This present application claims benefit of U.S. Provisional Patent Application Ser. No. 60/653,218, filed Feb. 15, 2005, which is incorporated herein by reference in its entirety and for all purposes.

### FIELD OF THE INVENTION

The present invention relates to new compounds, more particularly new dihydroindolyl methanones as α₁ₐ/α_{1d} adrenoreceptor modulators for the treatment of benign prostatic hypertrophy and/or lower urinary tract symptoms. The present invention also relates to pharmaceutical compositions comprising said new compounds, new processes to prepare these new compounds, to the use of these compounds as α₁ₐ/α_{1d} adrenoreceptor modulators and new uses as a medicine as well as method of treatments.

### RELATED ART

The adrenergic receptors (ARs), through which norepinephrine and epinephrine exert their biological activities, are targets for many therapeutically important drugs. The α₁-ARs play a dominant role in control of smooth muscle contraction and are important in control of blood pressure, nasal congestion, prostate function, and other processes (Harrison et al., Trends Pharmacol Sci; 1991; 62-67). The α₁-ARs were originally classified by pharmacological profiling into two subtypes, α₁ₐ and α_{1b} (Morrow and Creese, Mol. Pharmacol; 1986; 29: 231-330; Minneman et al., Mol. Pharmacol; 1988; 33:509-514). Three genes encoding different α₁-AR subtypes (α₁ₐ, α_{1b}, and α_{1d}) have been cloned for a number of species, including human (Schwinn et al., J. Biol Chem; 1990; 265: 8183-8189; Ramarao et al., J Biol Chem; 1992; 267:21936-21945; Bruno et al., Biochem Biophys Res Commun; 1991; 179: 1485-1490). These three cloned α₁-ARs are best differentiated from one another on the basis of the relative binding affinities of a series of antagonist compounds. There is general agreement that the α₁ₐ- and α_{1b}-ARs correspond to the pharmacologically defined α₁ₐ- and α_{1b}-ARs, while the functional role of the α_{1d}-AR is less clear, although it appears to mediate contraction of certain blood vessels (Goetz et al., Eur J Pharmacol; 1991; 272:R5-R6). Like other ARs, the α₁-ARs are members of the G-protein coupled receptor super family, and in most cells the primary functional response to activation of all α₁-AR subtypes is an increase in intracellular Ca²⁺.

Benign prostatic hyperplasia (BPH) is a non-malignant enlargement of the prostate and is the cause of lower urinary tract symptoms (LUTS) in a large segment of the elderly male population. Symptoms such as straining, hesitancy, dribbling, weak stream, and incomplete emptying are classified as voiding or obstructive symptoms. Obstructive symptoms are primarily due to pressure upon the urethra from the physical mass of the enlarged prostate gland (the static component) and the increased tone of the smooth muscle of the prostate stroma and bladder neck (the dynamic component) (Caine, J Urol; 1986; 136: 14). Irritative or storage symptoms associated with BPH are frequency, urgency, nocturia, dysuria, and burning sensation. Patients feel that these symptoms are more disturbing than the obstructive symptoms. As the urine flow is reduced, due to the bladder outlet obstruction, the wall around the bladder base thickens and becomes hyperactive.

Functional studies have established that prostate smooth muscle tone is maintained through α₁-ARs and that these receptors mediate the dynamic component of obstruction. α₁-AR antagonists have successfully been used to treat the obstructive symptoms associated with BPH (Jardin et al., Scientific Communications Int; 1998; pp 559-632). Furthermore, the α₁ₐ-AR subtype comprises the majority of α₁-ARs in human prostatic smooth muscle and has been shown to mediate contraction in this tissue. Originally introduced as antihypertensive agents, α₁-AR antagonists have become increasingly important in the management of BPH. α₁-AR antagonists reduce smooth muscle tone in the prostate and lower urinary tract, thereby relaxing the bladder outlet and increasing urinary flow. The major disadvantage of non-selective α₁-blockers is their adverse effect profile, particularly vasodilatation leading to dizziness, postural hypotension, asthenia, and occasionally syncope. For this reason, it would be desirable to block α₁-ARs in the lower urinary tract without antagonizing the α₁-ARs responsible for maintaining vascular tone.

A number of factors can be involved in lower urinary tract symptoms. Adrenergic stimulation of the bladder results in relaxation due to β-ARs, which dominate over contraction-mediating α₁-ARs. Bladder contraction is primarily mediated by muscarinic receptors. Some studies indicate that the contribution from α₁-ARs increases in hyperactive bladders due to bladder outlet obstruction or other conditions (Perlberg et al., Urology; 1982; 20:524-527); Restorick and Mundy, Br J Urol; 1989; 63: 32-35). However another study finds no change in α₁-AR receptor function between normal and hypertrophic bladder due to outlet obstruction (Smith and Chapple, Neurolog Urodyn; 1994; 12: 414-415). It remains unclear, which α₁-AR is dominant in the human bladder. One study reported a predominance of the α₁ₐ subtype mRNA in the bladder dome, base, and trigone (Walden et al., J Urol; 1997; 157: 414-415). Another report found that the α_{1d} subtype is present as 66% of the α₁-ARs at both the mRNA and protein levels, while the α₁ₐ subtype is present as 34% of the total, with no evidence of the α_{1b} subtype (Malloy et al., J Urol; 1998; 160: 937-943). Drugs that selectively antagonize only the α₁ₐ-AR subtype appear to have little effect upon the irritative symptoms of BPH. Ro-70004, a α₁ₐ subtype-selective compound was reported to be discontinued in clinical studies when it was found to have poor efficacy in treating these symptoms (Blue et al., Abstract 5^{th} International Consultation on BPH (June 25-28) 2000). α_{1d}-ARs may be involved in mediating the irritative symptoms; however, the location of these α_{1d}-ARs is unknown (Piascik and Perez, J Pharmacol Exp Ther; 2001; 298: 403410).

Studies have demonstrated Central Nervous Systems (CNS) inhibitory effects of α₁ antagonists upon the sympathetic and somatic outflow to the bladder in cats (Danuser and Thor, J Urol; 1995; 153: 1308-1312; Ramage and Wyllie, Eur J Pharmacol; 1995; 294: 645-650). Intrathecally administered doxazosin caused a decrease in micturition pressure in both normal rats and rats with bladder hypertrophy secondary to outlet obstruction (Ishizuka et al., Br J Pharmacol; 1996; 117:962-966). These effects may be due to a reduction in parasympathetic nerve activity in the spinal cord and ganglia. Other studies used spontaneously hypertensive rats, which have overactive bladders, to demonstrate that α₁-AR antagonism only given intrathecally caused a return to normal micturition (Persson et al., Am J Physiol; 1998; 275:R₁₃₆₆-1373, Steers et al. 1999; Exp Physiol; 84:137-147.). Antagonists administered intra-arterially near the bladder, or ablation of peripheral noradrenergic nerves, had no effect upon the bladder overactivity in these animals, indicating that α₁-ARs in the spinal cord control the bladder activity. Spinal α₁-ARs may be important targets for pharmacological treatment of BPH symptoms in humans as well. All three α₁-AR subtype mRNAs are found throughout the human spinal cord, however the α_{1d} subtype mRNA is present at twice the level of the other subtypes, particularly in the ventral sacral motor neurons and autonomic parasympathetic pathways. (Stafford-Smith et al., Mol Brain Res; 1998; 63:234-261). There may be clinical advantages to the pharmacological blockade of the α_{1d}-ARs in the CNS in reducing BPH symptoms.

Antagonism of α_{1d}-ARs in the CNS and bladder may be an important activity in reducing the irritative or filling symptoms of BPH and improving patient symptom scores. Tamsulosin (Flomax®, Yamanuchi and Boehringer Ingelheim) is a α₁-AR antagonist, which is about 15-fold selective for the α₁ₐ and α_{1d} subtypes over the α_{1b} subtype. Large clinical trials of BPH patients with tamsulosin showed improvement in both obstructive and irritative symptoms, however, cardiovascular and erectile dysfunction side effects were seen (Abrams et al. Br J Urol; 1995; 76:325-336; Chapple et al., Eur Urol; 1996; 29:155-167; Lepor, Urology; 1998; 51:892-900). Patients treated with non-selective α₁ antagonists also have improvement in both obstructive and irritative symptoms, although the risk of vascular side effects is greater. Generally, the α₁ₐ subtype predominates in arteries at the mRNA and protein levels, while all three subtypes are found in veins. The particular vessel bed is important in that the α₁ₐ is the subtype found primarily in the splanchnic and coronary arteries, while the α_{1d} subtype is the predominant subtype found in the aorta. The α₁-AR subtypes in the vasculature have been found to change with age. Contraction of the mammary artery is mediated by both α₁ₐ and α_{1b} subtypes. The number of α₁ receptors in the mammary artery doubles with age; however, the α_{1b} subtype increases to a greater extent than the α₁ₐ subtype (Raudner et al., Circulation; 1999; 100:2336-2343). The α_{1b} subtype may play a greater role in vascular tone in elderly patients. This suggests that an α₁ₐ and α_{1d}-selective antagonist may have less effects upon the vasculature in elderly BPH patients, resulting in fewer cardiovascular side effects than are seen with non-selective α₁ antagonists, but provide relief from both obstructive and irritative symptoms.

A uroselective, cardiovascular-sparing α₁-AR antagonist would be expected to provide symptomatic relief of BPH comparable to currently marketed non-selective agents such as terazosin/Hytrin®, doxazosin/Cardura®, alfuzosin/Xatral®/Uroxatral® and weakly selective tamsulosin/Flomax®/Harnal®, without the undesirable side effects of postural hypotension, dizziness, and syncope. Ejaculatory dysfunction, or retrograde ejaculation, is a side effect seen in 10 to 35% of patients using tamsulosin (Lepor, Urology; 1998; 51:901-906; Andersson and Wyllie, Brit J Urol Int; 2003; 92:876-877). This activity has been attributed to tamsulosin antagonism at the 5-HT₁ₐ receptor. This often leads to discontinuation of treatment. Furthermore, the non-selective α₁-AR antagonists and tamsulosin are contraindicated for use in conjunction with PDE inhibitors. There is likely to be high comorbidity between LUTS and erectile dysfunction patients. Patients being treated for LUTS with the current α₁-AR blockers will find that they are excluded from using PDE inhibitors. An α₁-AR antagonist with a receptor subtype binding profile, which is selective for the α₁ₐ and α_{1d}, subtypes, but with relatively little antagonism of the α_{1b} subtype may effectively treat both obstructive and irritative symptoms of BPH. Such a compound is likely to have a low cardiovascular side effect profile and allow for use in conjunction with PDE inhibitors. Also low binding activity at the 5-HT₁ₐ receptor is likely to reduce the incidence of ejaculatory side effects.

LUTS also develop in women of a certain age. As in men, LUTS in women include both filling symptoms such as urgency, incontinence and nocturnia, and voiding symptoms such as weak stream, hesitancy, incomplete bladder emptying and abdominal straining. The presence of this condition both in men and women suggests that at least part of the aetiology may be similar in the two sexes.

A (2,3-dihydro-indol-1-yl)-{4-[4-(2-isopropoxy-phenyl)-piperazine-1-ylmethyl]-phenyl}-methanone compound has been described as an antipsychotic having α₁-AR activity (The Journal of Medicinal Chemistry, Vol. 41 (12), pp 1997-2009).

Accordingly, there is a need to provide dual selective α₁ₐ/α_{1d} adrenoreceptor modulators, particularly dual selective α₁ₐ/α_{1d} adrenoreceptor antagonists, in other words compounds that interact both with the α₁ₐ and α_{1d} receptor but do not interact (or at least interact substantially less) with the α_{1b} receptor. The compounds of this invention are believed to be more efficacious drugs mainly for BPH/LUTS patients, and at the same time these compounds should show less unwanted side effects than the existing pharmaceuticals.

### SUMMARY OF THE INVENTION

The present invention provides a compound of Formula (I) 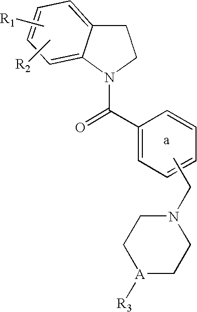
and pharmaceutically acceptable forms thereof, wherein
“a” represents a point of attachment selected from the 3 or 4 position on the phenyl ring relative to the point of attachment of the methanone group for the compound of Formula (I),
“A” is a ring atom selected from CH or N,
R₁ is one substituent selected from the group consisting of hydrogen, —N—, halogen and nitro, wherein —N— is substituted with two substituents independently selected from the group consisting of hydrogen, C₁₋₈alkyl, C₁₋₈alkyl(C₁₋₈alkoxy), C(O)(R^{A}), C(O)O(R^{A}), C(O)NH₂, C(O)NH(C₁₋₈alkyl), C(O)N(C₁₋₈alkyl)₂, C(O)NH(R^{A}), C(O)N(R^{A})₂, C(O)NH(C₁₋₈alkyl-R^{A}), C(O)N(C₁₋₈-R^{A})₂, C(S)NH(R^{A}), C(S)N(R^{A})₂, C(S)NH(C₁₋₈alkyl-R^{A}), C(S)N(C₁₋₈alkyl-R^{A})₂, SO₂(C₁₋₈alkyl), SO₂(R^{A}), SO₂NH₂, SO₂NH(C₁₋₈alkyl), SO₂N(C₁₋₈alkyl)₂, SO₂NH(R^{A}), C₁₋₈alkyl(R^{A}) and R^{A},
R₂ is one substituent selected from the group consisting of hydrogen, —SO₂— and R^{A}, wherein —SO₂— is substituted with C₁₋₈alkyl, NH₂, NH(C₁₋₈alkyl) or N(C₁₋₈alkyl)₂,
R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), NH₂, NH(C₁₋₈alkyl), N(C₁₋₈alkyl)₂, halogen, hydroxy, C(O)NH₂, C(O)NH(C₁₋₈alkyl), C(O)N(C₁₋₈alkyl)₂, NHC(O)H and NHC(O)(C₁₋₈alkyl),
R₃ is selected from the group consisting of C₁₋₈alkyl(R^{B}), —C(O)(C₁₋₈alkoxy) and R^{B}, and
R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), C₁₋₈alkoxy(hydroxy), cyano, halogen and hydroxy,
with the proviso that (2,3-dihydro-indol-1-yl)-{4-[4-(2-isopropoky-phenyl)-piperazine-1-ylmethyl]-phenyl}-methanone is not included as a compound of Formula (I).

Examples of the invention include pharmaceutical compositions comprising a therapeutically effective amount of any of the compounds of Formula (I) described in the present application and a pharmaceutical acceptable carrier.

An example of the invention is a pharmaceutical composition made by combining any of the compounds of Formula (I) described in the present application and a pharmaceutically acceptable carrier.

Another illustration of the invention is a process for making a pharmaceutical composition comprising combining any of the compounds described in the present application and a pharmaceutically acceptable carrier.

The present invention further provides a method for treating a patient suffering from a disease or disorder mediated by dual selective α₁ₐ/α_{1d} adrenoreceptor modulators comprising administering to the patient an effective amount of the compound of Formula (I) and pharmaceutically acceptable forms thereof.

An example of the invention includes a method for treating a patient suffering from a disease or disorder mediated by dual selective α₁ₐ/α_{1d} adrenoreceptor modulators comprising administering to the patient an effective amount of a compound of Formula (II). 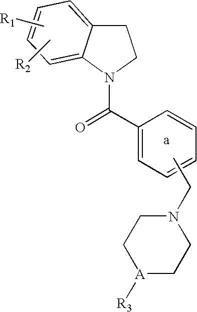
and pharmaceutically acceptable forms thereof, wherein
“a” represents a point of attachment selected from the 3 or 4 position on the phenyl ring relative to the point of attachment of the methanone group for the compound of Formula (II),
“A” is a ring atom selected from CH or N,
R₁ is one substituent selected from the group consisting of hydrogen, —N—, halogen and nitro, wherein —N— is substituted with two substituents independently selected from the group consisting of hydrogen, C₁₋₈alkyl, C₁₋₈alkyl(C₁₋₈alkoxy), C(O)(R^{A}), C(O)O(R^{A}), C(O)NH₂, C(O)NH(C₁₋₈alkyl), C(O)N(C₁₋₈alkyl)₂, C(O)NH(R^{A}), C(O)N(R^{A})₂, C(O)NH(C₁₋₈alkyl-R^{A}), C(O)N(C₁₋₈alkyl-R^{A})₂, C(S)NH(R^{A}), C(S)N(R^{A})₂, C(S)NH(C₁₋₈alkyl-R^{A}), C(S)N(C₁₋₈alkyl-R^{A})₂, SO₂(C₁₋₈alkyl), SO₂(R^{A}), SO₂NH₂, SO₂NH(C₁₋₈alkyl), SO₂N(C₁₋₈alkyl)₂, SO₂NH(R^{A}), C₁₋₈alkyl(R^{A}) and R^{A},
R₂ is one substituent selected from the group consisting of hydrogen, —SO₂— and R^{A}, wherein —SO₂— is substituted with C₁₋₈alkyl, NH₂, NH(C₁₋₈alkyl) or N(C₁₋₈alkyl)₂,
R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), NH₂, NH(C₁₋₈alkyl), N(C₁₋₈alkyl)₂, halogen, hydroxy, C(O)NH₂, C(O)NH(C₁₋₈alkyl), C(O)N(C₁₋₈alkyl)₂, NHC(O)H and NHC(O)(C₁₋₈alkyl),
R₃ is selected from the group consisting of C₁₋₈alkyl(R^{B}), —C(O)(C₁₋₈alkoxy) and R^{B}, and
R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), C₁₋₈alkoxy(hydroxy), cyano, halogen and hydroxy.

It should be understood that pharmaceutically acceptable forms for compounds described and listed herein are meant to include all hydrates, solvates, polymorphs and pharmaceutically acceptable salts thereof. It should also be understood that unless otherwise indicated compounds of Formula (I) and Formula (II) are meant to comprise the stereochemically isomeric forms thereof.

An aspect of the invention is directed to methods for treating or preventing a disease or disorder mediated by dual selective α₁ₐ/α_{1d} adrenoreceptor modulators, more particularly dual selective α₁ₐ/α_{1d} adrenoreceptor antagonists such as, but not limited to, contractions of the prostate, bladder and other organs of the lower urinary tract without substantially affecting blood pressure. In this aspect, the method comprises administering the dual selective α₁ₐ/α_{1d} adrenoreceptor modulator compounds of the present invention or a pharmaceutically acceptable form thereof to a patient suffering from contractions of the bladder and other organs of the lower urinary tract in an amount effective for the particular use.

Another aspect of the present invention is to provide a method for treating a patient suffering from Benign Prostatic Hyperplasia (BPH). In this aspect, the method comprises administering an effective amount of the modulator compounds of the present invention or a pharmaceutically acceptable form thereof to a patient suffering from BPH.

Another aspect of the present invention is to provide a method for treating a patient suffering from lower-urinary-tract-symptoms (LUTS), which include, but are not limited to, filling symptoms, urgency, incontinence and nocturia, as well as voiding problems such as weak stream, hesitancy, intermittency, incomplete bladder emptying and abdominal straining. In this aspect, the method comprises administering an effective amount of the modulator compounds of the present invention or a pharmaceutically acceptable form thereof to a patient suffering from LUTS.

A further aspect of the present invention is the use of the modulator compounds of the present invention or a pharmaceutically acceptable form thereof as a medicament. In this aspect, the use of the modulator compound or pharmaceutically acceptable form thereof includes the manufacture of a medicament for treating BPH and/or LUTS.

In another aspect of the present invention, the method for treating a patient suffering from BPH and/or LUTS includes administering an effective amount of a combination product comprising a modulator compound of the present invention in combination with a BPH and/or LUTS therapeutic agent. The BPH and/or LUTS therapeutic agent includes a 5α-reductase agent (such as finasteride or durasteride and the like or mixtures thereof), a NK-1 inhibitor, an anti-androgen receptor agonist, an androgen receptor antagonist, a selective androgen receptor modulators, a PDE inhibitor, a urinary incontinence drugs (e.g. anti-muscarinics) or a 5HT-receptor modulator.

### DETAILED DESCRIPTION OF THE INVENTION

An example of the present invention includes a compound of Formula (I) and pharmaceutically acceptable forms thereof, wherein R₁ is one substituent selected from the group consisting of hydrogen, —N—, halogen and nitro, wherein —N— is substituted with two substituents independently selected from the group consisting of hydrogen, C(O)(R^{A}), C(O)O(R^{A}), C(O)NH₂, C(O)NH(R^{A}), C(O)N(R^{A})₂, C(O)NH(C₁₋₈alkyl-R^{A}), C(O)N(C₁₋₈alkyl-R^{A})₂, C(S)NH(R^{A}), C(S)N(R^{A})₂, C(S)NH(C₁₋₈alkyl-R^{A}), C(S)N(C₁₋₈alkyl-R^{A})₂, SO₂(C₁₋₈alkyl), SO₂(R^{A}), SO₂NH², SO₂NH(R^{A}), C₁₋₈alkyl(R^{A}) and R^{A}.

Another example of the present invention includes a compound of Formula (I) and pharmaceutically acceptable forms thereof, wherein R₁ is one substituent selected from the group consisting of hydrogen, —N—, halogen and nitro, wherein —N— is substituted with two substituents independently selected from the group consisting of hydrogen, C(O)(R^{A}), C(O)O(R^{A}), C(O)NH₂, C(O)NH(R^{A}), C(S)NH(R^{A}), SO₂(C₁₋₈alkyl), SO₂(R^{A}), SO₂NH₂, C₁₋₈alkyl(R^{A}) and R^{A}.

An example of the present invention includes a compound of Formula (I) and pharmaceutically acceptable forms thereof, wherein R₂ is one substituent selected from the group consisting of hydrogen, —SO₂— and R^{A}, wherein —SO₂— is substituted with C₁₋₈alkyl or N(C₁₋₈alkyl)₂.

An example of the present invention includes a compound of Formula (I) and pharmaceutically acceptable forms thereof, wherein R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, NH₂, NH(C₁₋₈alkyl), N(C₁₋₈alkyl)₂, halogen, hydroxy and NHC(O)(C₁₋₈alkyl).

Another example of the present invention includes a compound of Formula (I) and pharmaceutically acceptable forms thereof, wherein R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, N(C₁₋₈alkyl)₂, halogen and NHC(O)(C₁₋₈alkyl).

An example of the present invention includes a compound of Formula (I) and pharmaceutically acceptable forms thereof, wherein R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(halogen)₁₋₃, C₁₋₈alkoxy(halogen)₁₋₃, cyano, halogen and hydroxy.

Another example of the present invention includes a compound of Formula (I) and pharmaceutically acceptable forms thereof, wherein R^{B} is aryl optionally substituted with one to three substituents independently selected from the group consisting of C₁₋₁₀alkyl, C₁₋₈alkoxy, C₁₋₈alkyl(halogen)₁₋₃, C₁₋₈alkoxy(halogen)₁₋₃, cyano, halogen and hydroxy.

Another example of the present invention includes a compound of Formula (I) and pharmaceutically acceptable forms thereof, wherein R^{B} is aryl optionally substituted with one to three substituents independently selected from the group consisting of C₁₋₈alkoxy, C₁₋₈alkoxy(halogen)₁₋₃, cyano and hydroxy.

An example of the present invention includes a compound of Formula (Ia):

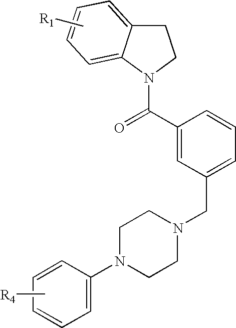

and pharmaceutically acceptable forms thereof, wherein R₁, and R₄ are dependently selected from:

| Cpd | R₁ | R₄ |
|---|---|---|
| 1 | 5-NH₂ | 2-OCH(CH₃)₂ |
| 2 | 5-NHSO₂-naphthalen-2-yl | 2-OCH(CH₃)₂ |
| 3 | 5-NHSO₂-5-N(CH₃)₂-naphthalen-1-yl | 2-OCH(CH₃)₂ |
| 4 | 5-NHC(O)O-4-CH₃-phenyl | 2-OCH(CH₃)₂ |
| 5 | 6-NH₂ | 2-OCH(CH₃)₂ |
| 6 | 6-NHCH₂-2,6-F₂-phenyl | 2-OCH(CH₃)₂ |
| 7 | 6-NHCH₂-2,6-(OCH₃)₂-phenyl | 2-OCH(CH₃)₂ |
| 8 | 5-NHCH₂-4-CF₃-phenyl | 2-OCH(CH₃)₂ |
| 9 | 5-NHCH₂-2,6-F₂-phenyl | 2-OCH(CH₃)₂ |
| 10 | 6-NHCH₂-4-(CH₂)₇CH₃-phenyl | 2-OCH(CH₃)₂ |
| 11 | 6-NHCH₂-4-CH(OCH₂CH₃)₂-phenyl | 2-OCH(CH₃)₂ |
| 12 | 6-NHC(O)O-4-CH₃-phenyl | 2-OCH(CH₃)₂ |
| 13 | 6-NHC(S)NH-phenyl | 2-OCH(CH₃)₂ |
| 14 | 5-NHC(O)NH-2,4-Cl₂-phenyl | 2-OCH(CH₃)₂ |
| 15 | 6-NHSO₂-4-NHC(O)CH₃-phenyl | 2-OCH(CH₃)₂ |
| 16 | 6-NH-1,2,3,4-tetrahydro-naphthalen-2-yl | 2-OCH(CH₃)₂ |
| 17 | 5-NHSO₂NH₂ | 2-OCH(CH₃)₂ |
| 18 | 6-NHSO₂-5-Cl-3-CH₃-benzo[b]thien-2-yl | 2-OCH(CH₃)₂ |
| 19 | 5-NHSO₂-4-NHC(O)CH₃-phenyl | 2-OCH(CH₃)₂ |
| 20 | 6-NHSO₂NH₂ | 2-OCH(CH₃)₂ |
| 21 | 5-NHC(O)NH-phenyl | 2-OCH(CH₃)₂ |
| 22 | 6-NHC(O)NH-phenyl | 2-OCH(CH₃)₂ |
| 23 | 5-NHCH₂-4-(CH₂)₇CH₃-phenyl | 2-OCH(CH₃)₂ |
| 24 | 5-NHCH₂-4-CH(OCH₂CH₃)₂-phenyl | 2-OCH(CH₃)₂ |
| 25 | 5-NHCH₂-2,6-(OCH₃)₂-phenyl | 2-OCH(CH₃)₂ |
| 26 | 5-NHC(O)-2,6-(OCH₃)₂-phenyl | 2-OCH(CH₃)₂ |
| 27 | 5-NHCH₂-benzo[1,3]dioxol-5-yl | 2-OCH(CH₃)₂ |
| 28 | 6-NHC(O)-2,6-(OCH₃)₂-phenyl | 2-OCH(CH₃)₂ |
| 29 | 6-NH-4,7-(OCH₃)₂-indan-2-yl | 2-OCH(CH₃)₂ |
| 30 | H | 2-OCH₃ |
| 31 | H | 2-CN |
| 32 | 6-NHSO₂-5-N(CH₃)₂-naphthalen-1-yl | 2-OCH(CH₃)₂ |
| 40 | 6-NHC(O)-2,6-F₂-phenyl | 2-OCH(CH₃)₂ |
| 41 | 6-NHCH₂-benzo[1,3]dioxol-5-yl | 2-OCH(CH₃)₂ |
| 42 | 6-NHC(O)-4-OCH₃-phenyl | 2-OCH(CH₃)₂ |
| 43 | 6-N[C(O)NH-2,6-F₂-phenyl]₂ | 2-OCH(CH₃)₂ |
| 44 | 6-NHCH₂-2-F-phenyl | 2-OCH(CH₃)₂ |
| 45 | 6-NHSO₂-2,6-F₂-phenyl | 2-OCH(CH₃)₂ |
| 46 | 6-NHSO₂-4-CH₃-phenyl | 2-OCH(CH₃)₂ |
| 47 | 6-NHSO₂CH₂CH₃ | 2-OCH(CH₃)₂ |
| 48 | 6-NHC(O)-4-F-phenyl | 2-OCH(CH₃)₂ |
| 49 | 6-NHC(O)NH₂ | 2-OCH(CH₃)₂ |
| 63 | H | 2-OCH₂CF₃ |
| 65 | H | 2-OCH₂CH(CH₃)₂ |
| 66 | 5-Br | 2-OCH₂CH(CH₃)₂ |
| 67 | 5-Cl | 2-OCH₂CH(CH₃)₂ |
| 68 | 5-F | 2-OCH₂CH(CH₃)₂ |
| 69 | H | 2-OCH₂CH₃ |
| 73 | 6-NHC(O)NH-2,6-F₂-phenyl | 2-OCH(CH₃)₂ |

An example of the present invention includes a compound of Formula (Ib):

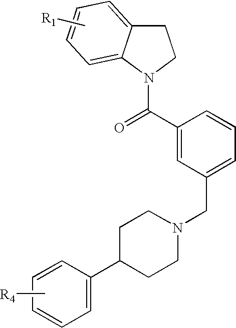

and pharmaceutically acceptable forms thereof, wherein R₁ and R₄ are dependently selected from:

| Cpd | R₁ | R₄ |
|---|---|---|
| 51 | H | 2-OCH(CH₃)₂ |
| 57 | 6-NHCH₂-2-F-phenyl | 2-OCH(CH₃)₂ |
| 58 | 6-NHSO₂-2,6-F₂-phenyl | 2-OCH(CH₃)₂ |
| 59 | 6-NHCH₂-2-F-phenyl | 2-OCH₃ |
| 60 | 6-NHC(O)NH-2,6-F₂-phenyl | 2-OCH(CH₃)₂ |
| 61 | 6-NHSO₂-2,6-F₂-phenyl | 2-OCH₃ |

An example of the present invention includes a compound of Formula (Ic):

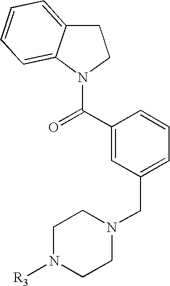

and pharmaceutically acceptable forms thereof, wherein R₃ is selected from:

| Cpd | R₃ |
|---|---|
| 54 | C(O)OC(CH₃)₃ |
| 56 | CH₂-2-OCH(CH₃)₂-phenyl |
| 64 | CH₂-2-OCH₃-phenyl |

An example of the present invention includes a compound of Formula (Id):

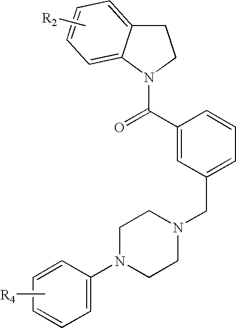

and pharmaceutically acceptable forms thereof, wherein R₂ and R₄ are dependently selected from:

| Cpd | R₂ | R₄ |
|---|---|---|
| 70 | 5-SO₂N(Me)₂ | 2-OCH₂CH(CH₃)₂ |
| 71 | 5-SO₂Me | 2-OCH₂CH(CH₃)₂ |
| 72 | 5-Ph | 2-OCH₂CH(CH₃)₂ |

An example of the present invention includes a compound of Formula (Ie):

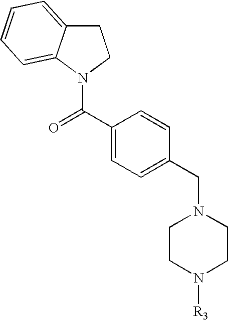

and pharmaceutically acceptable forms thereof, wherein R₃ is selected from:

| Cpd | R₃ |
|---|---|
| 52 | CH₂-2-OCH(CH₃)₂-phenyl |
| 53 | CH₂-2-OCH₃-phenyl |
| 55 | 2-OCH₂CF₃-phenyl |
| 62 | CH₂-2-OH-phenyl |

An example of the present invention includes a compound of Formula (If): 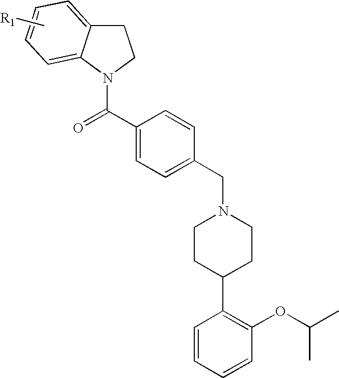
and pharmaceutically acceptable forms thereof, wherein R₁ is selected from:

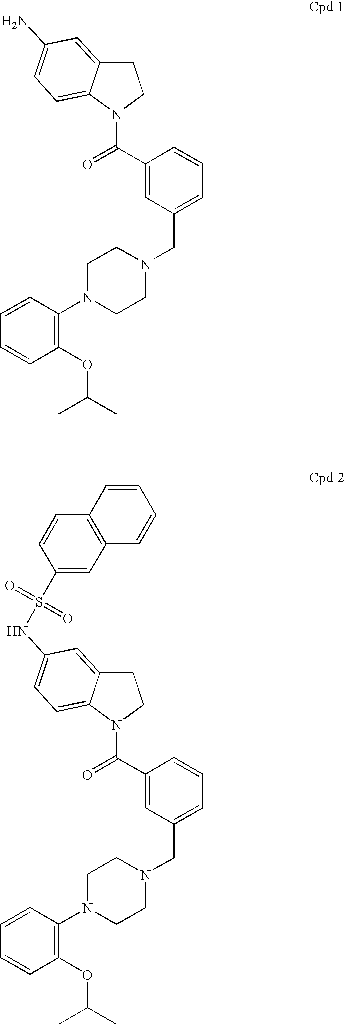

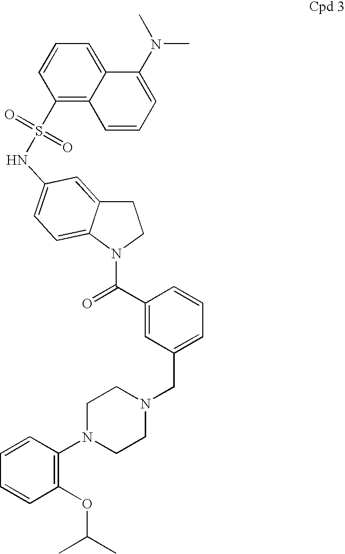

Another example of the present invention includes a compound selected from the group consisting of 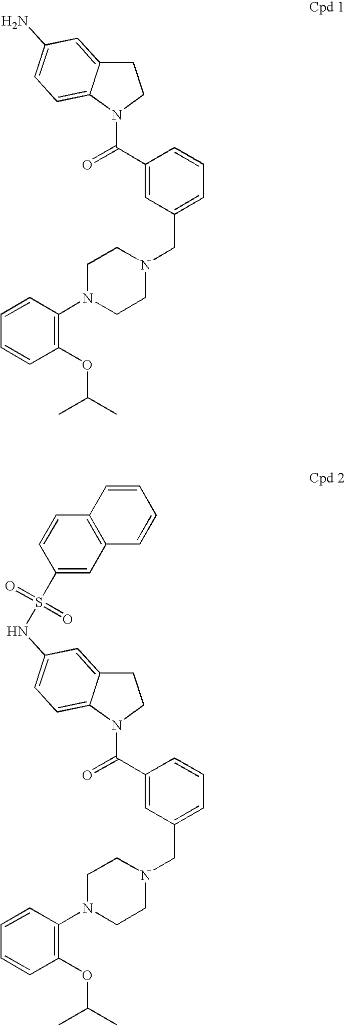 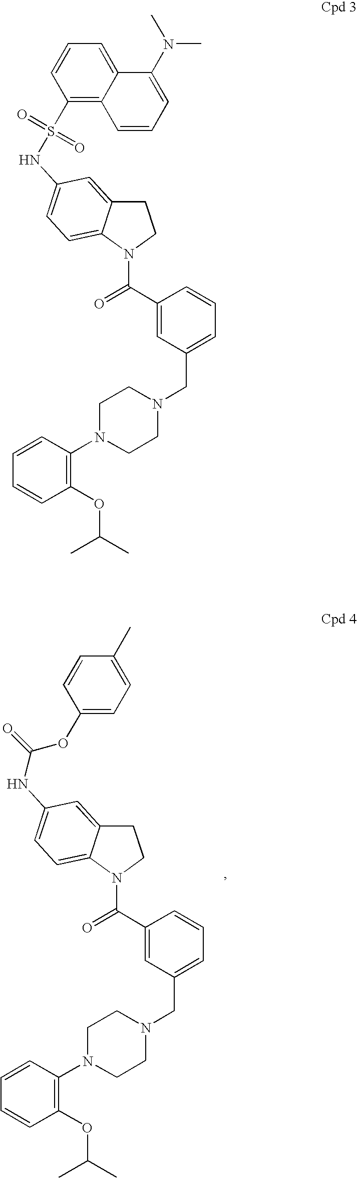 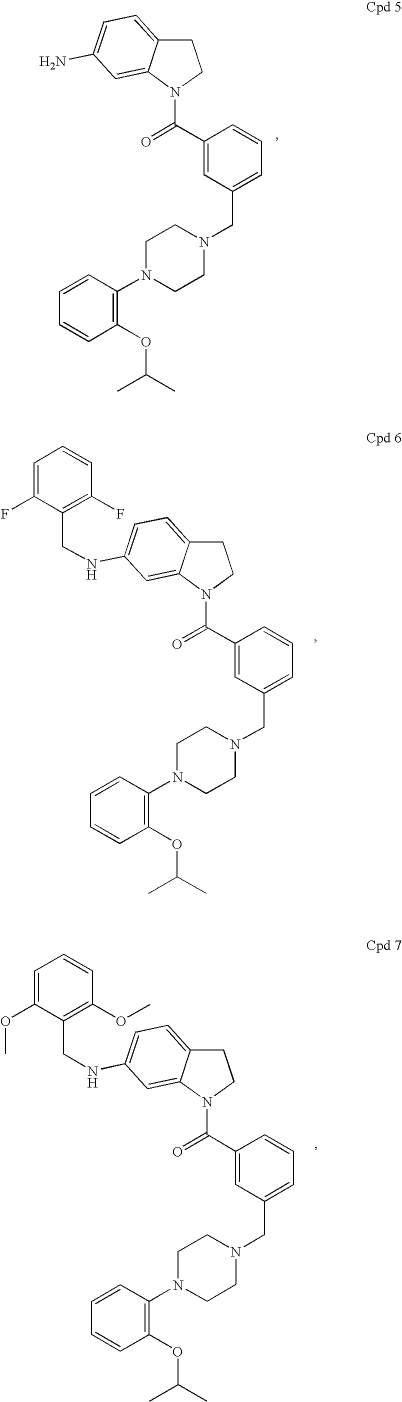 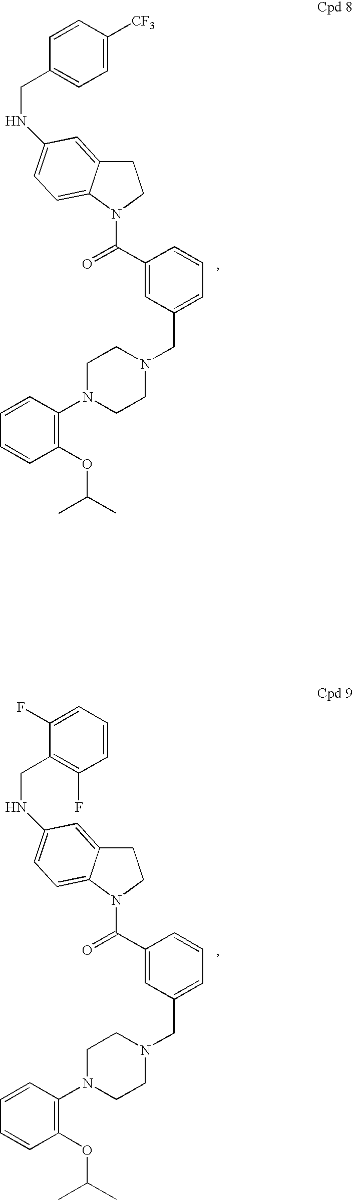 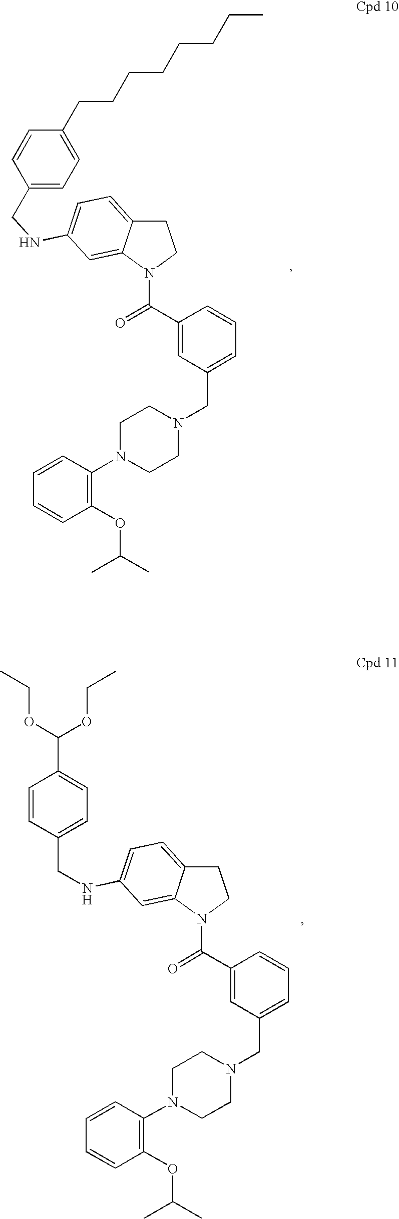 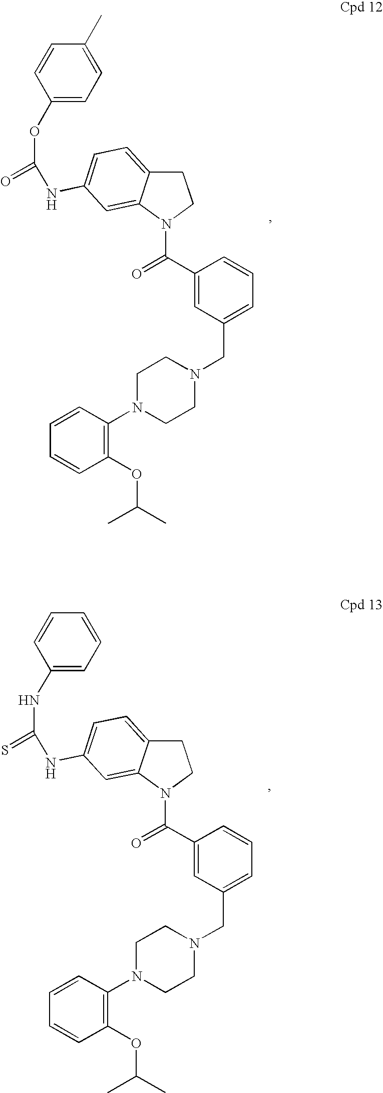 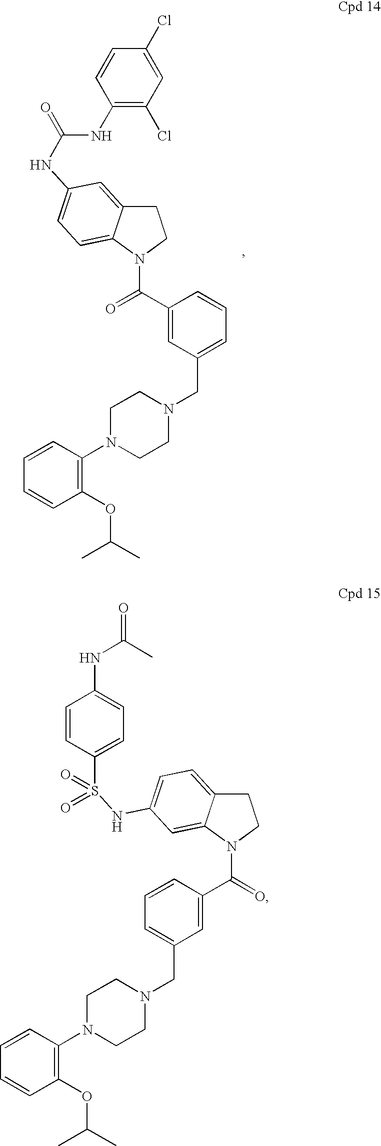 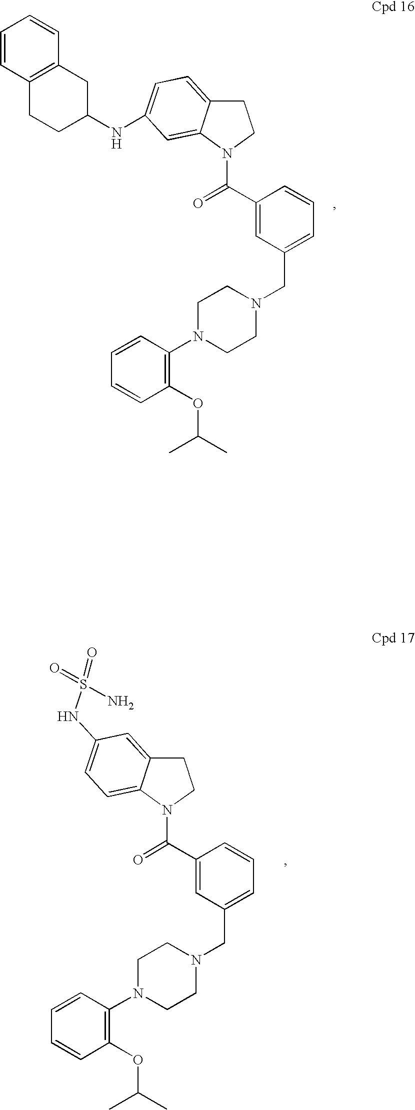 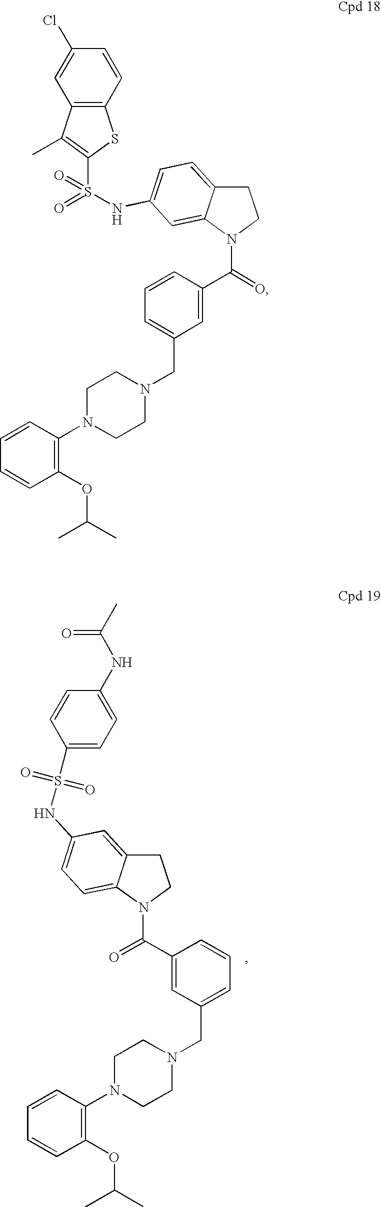 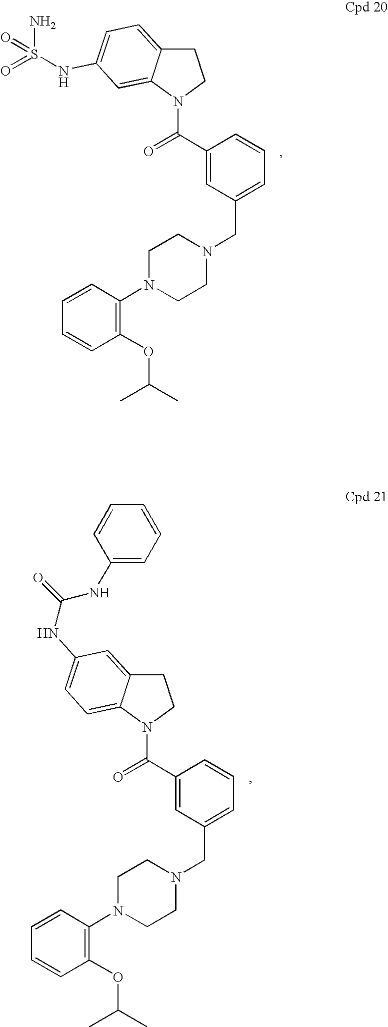 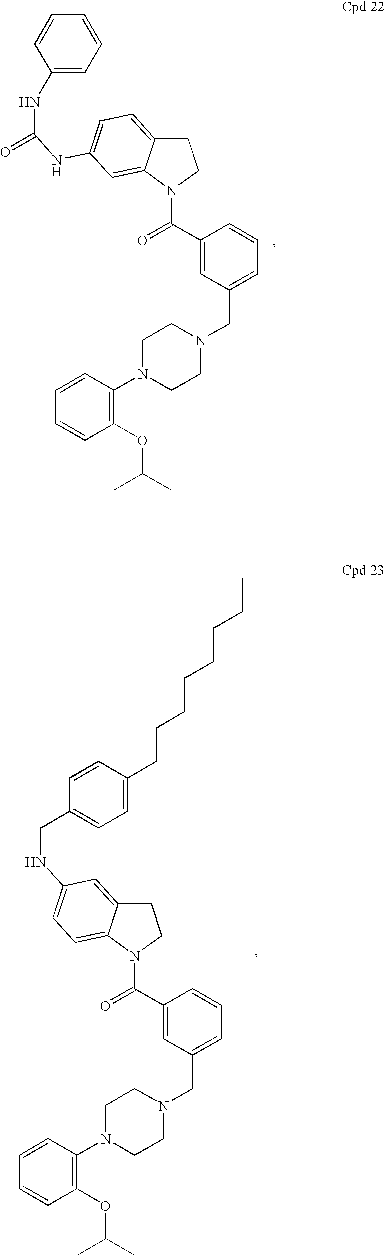 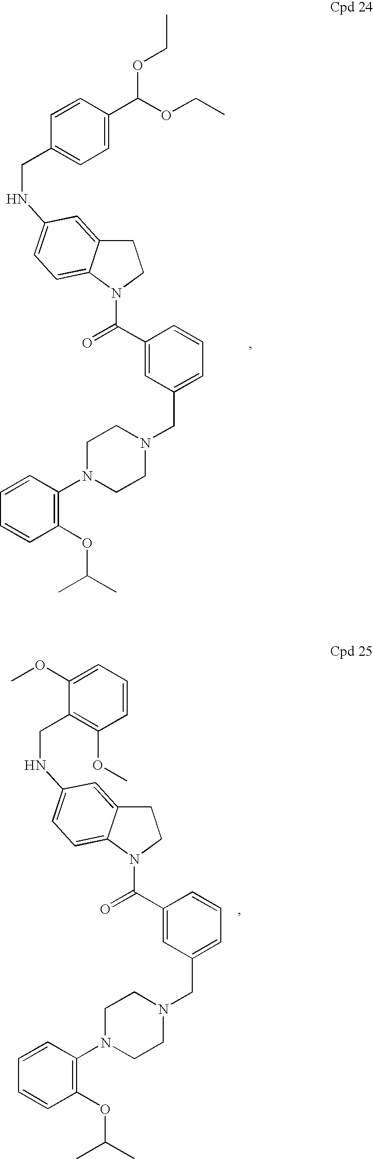 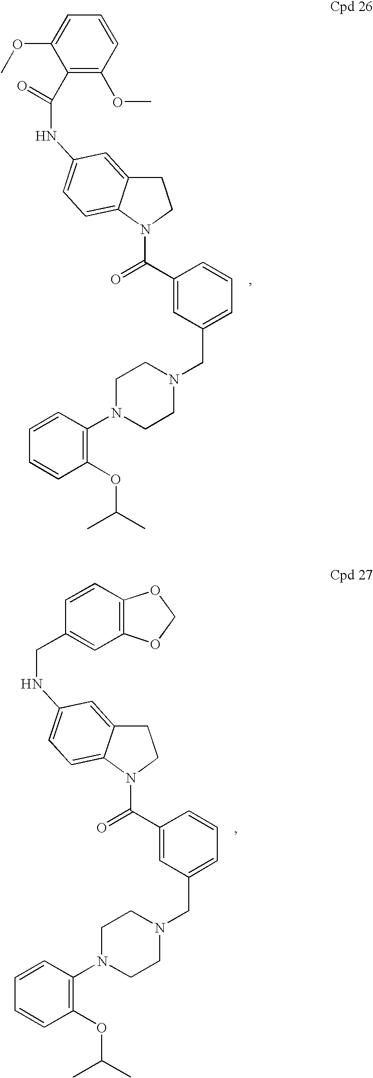 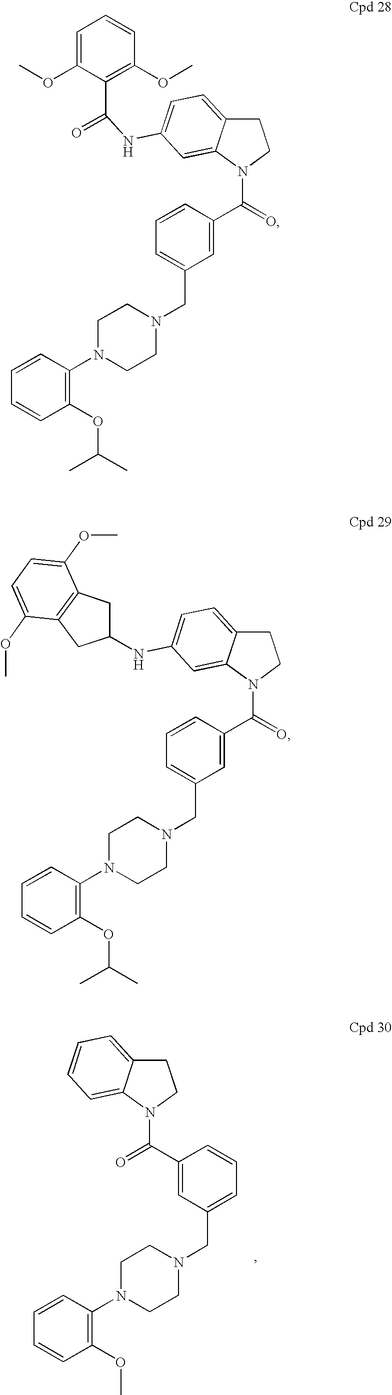 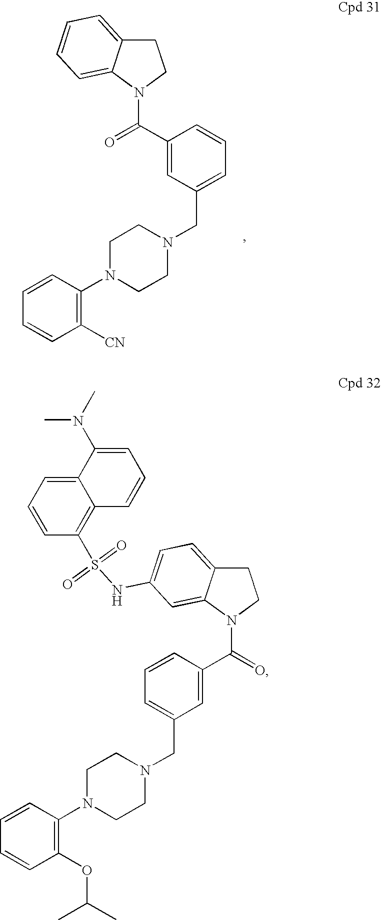 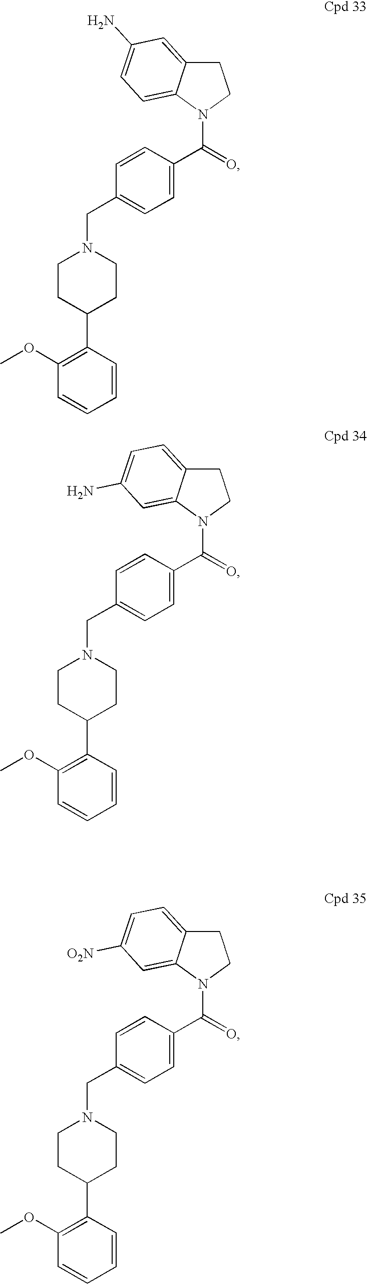 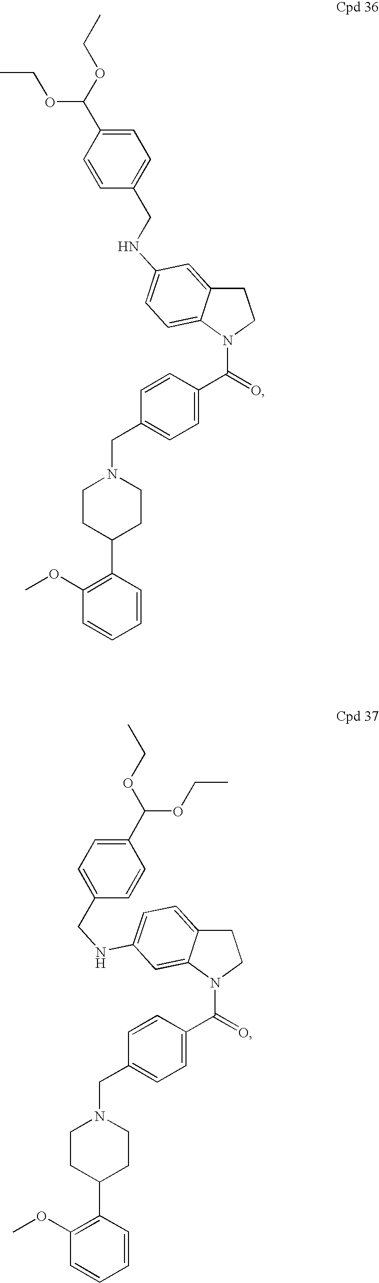 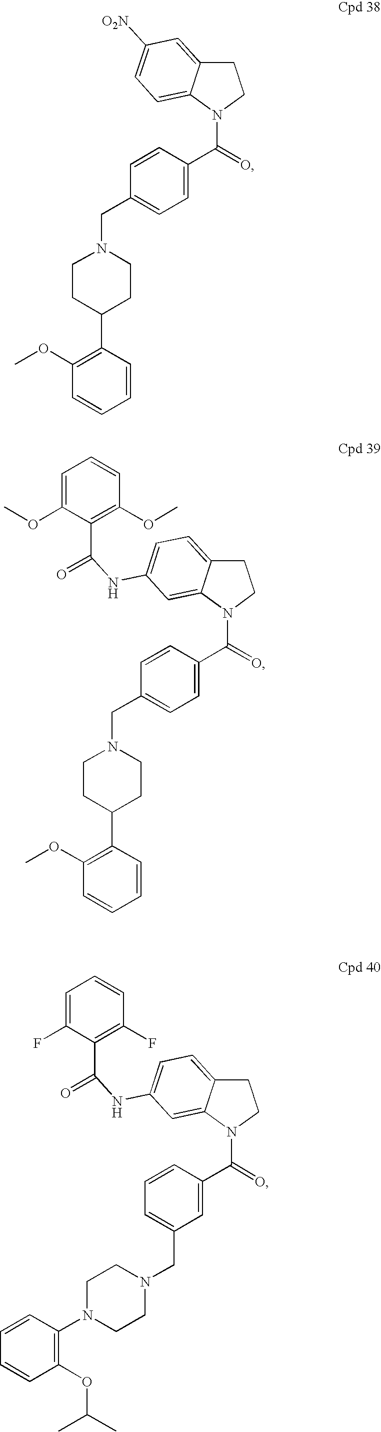 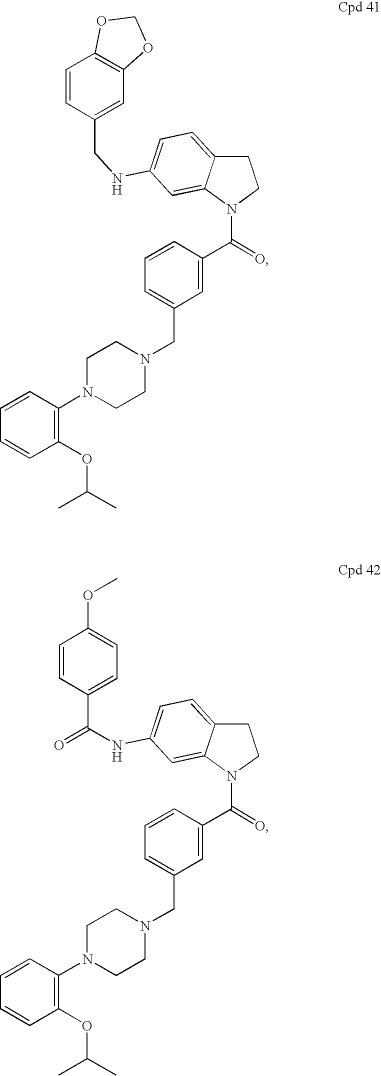 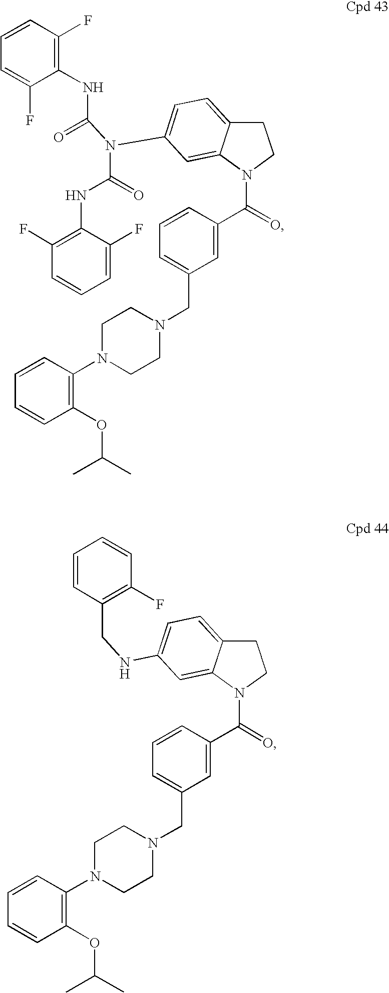 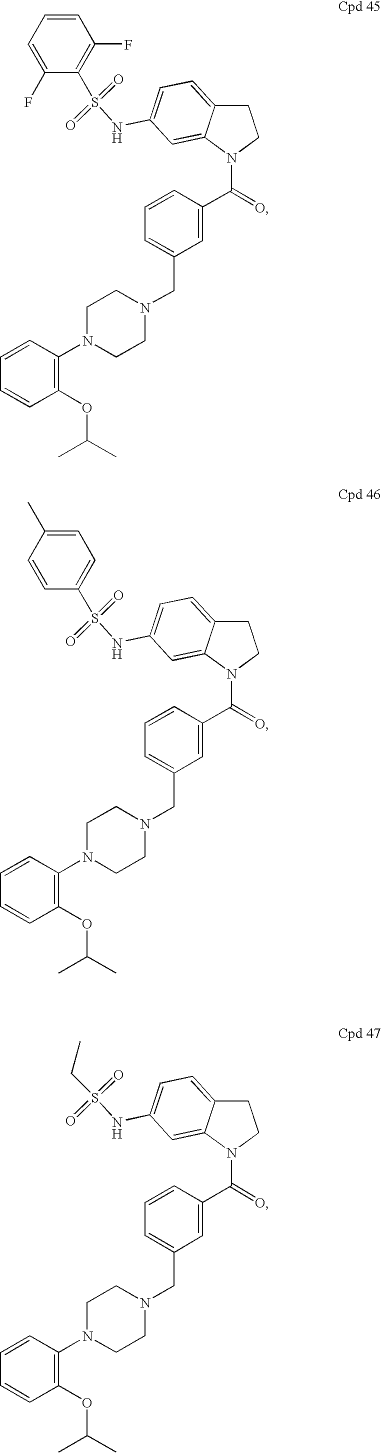 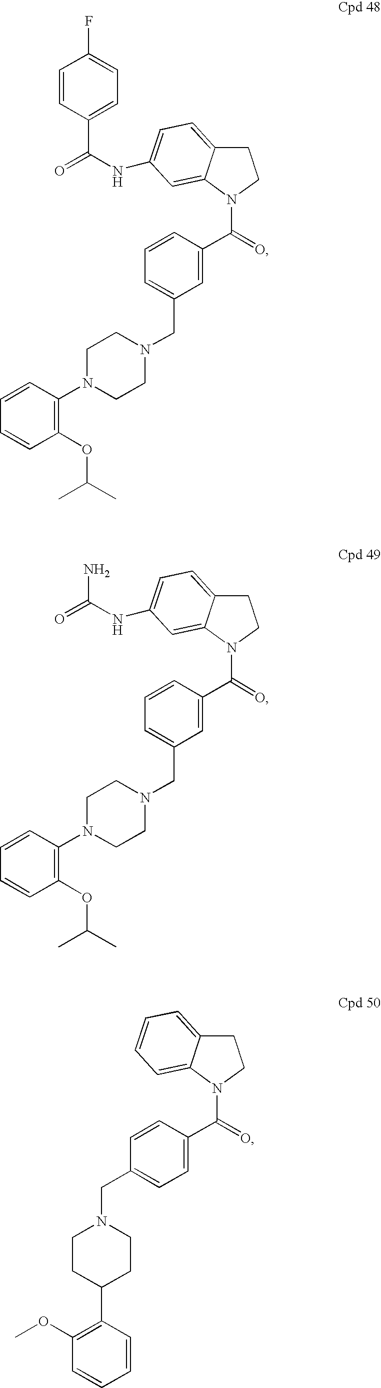 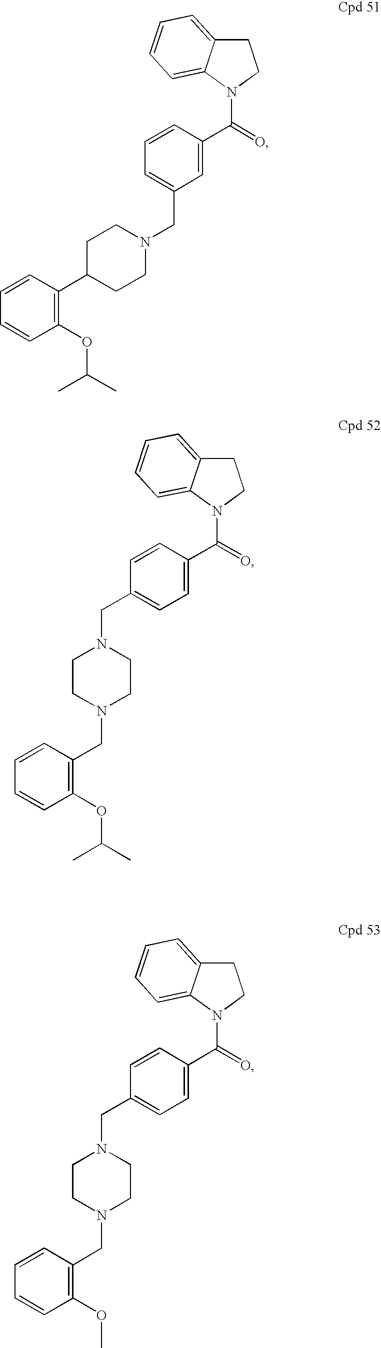 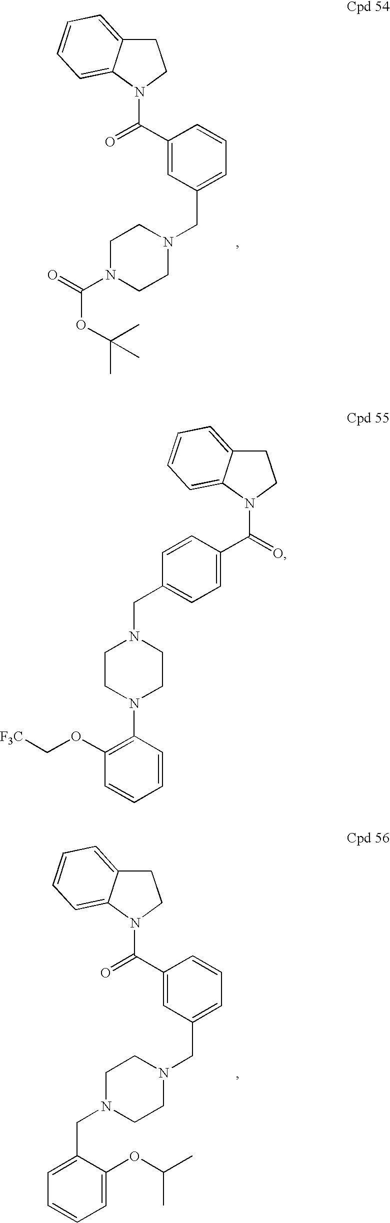 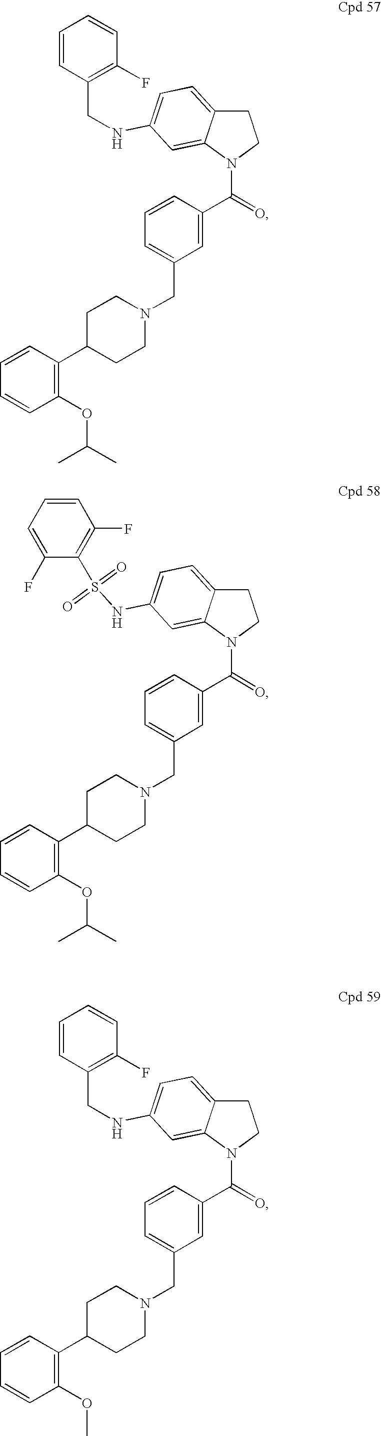 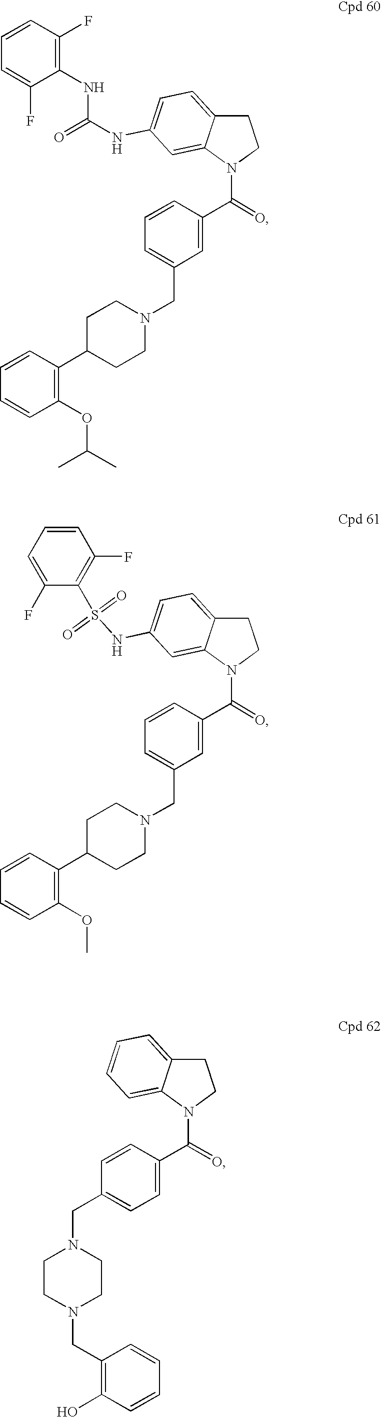 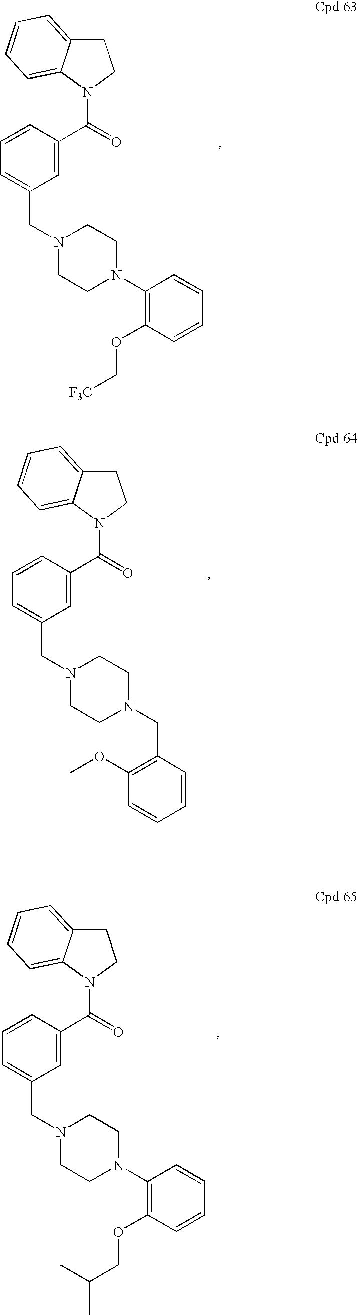 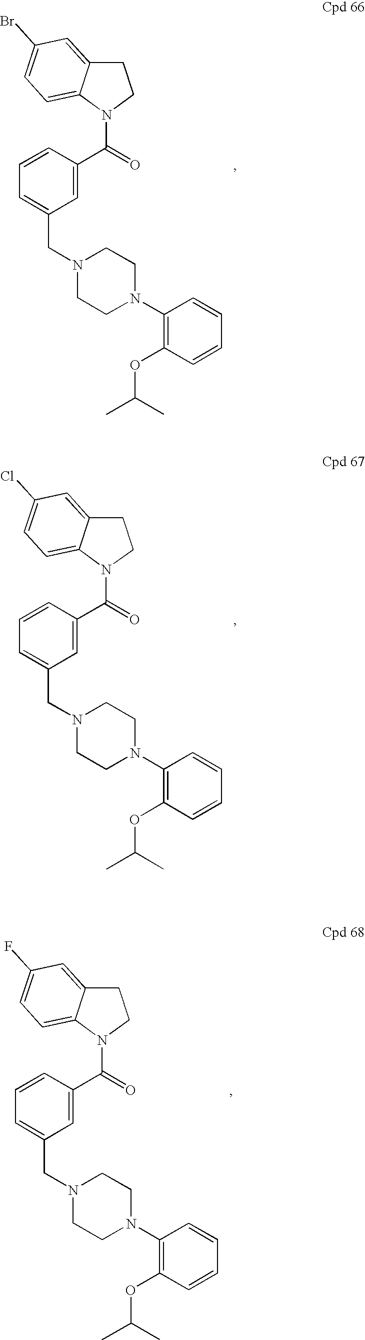 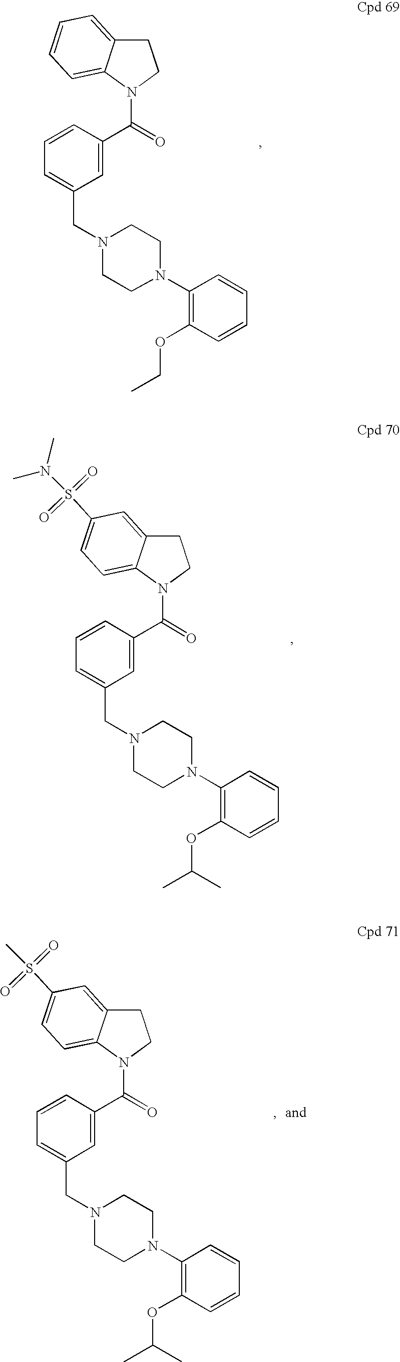 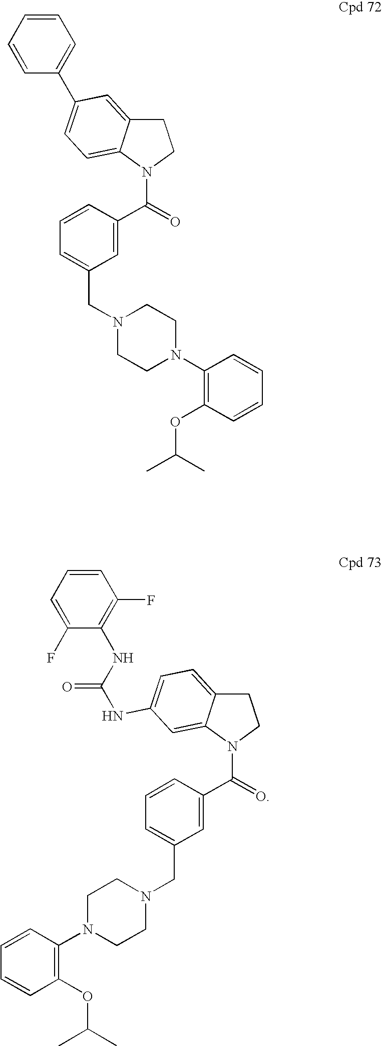
Compound Forms

The compounds of the present invention may be present in the form of pharmaceutically acceptable salts. For use in medicines, the “pharmaceutically acceptable salts” of the compounds of this invention refer to non-toxic acidic/anionic or basic/cationic salt forms.

Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, fumaric acid, maleic acid, succinic acid, acetic acid, benzoic acid, citric acid, tartaric acid, carbonic acid or phosphoric acid.

Furthermore when the compounds of the present invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts. Thus, representative pharmaceutically acceptable salts include the following: acetate, benzenesulfonate, benzoate, bicarbonate, bisulfate, bitartrate, borate, bromide, calcium, camsylate (or camphosulphonate), carbonate, chloride, clavulanate, citrate, dihydrochloride, edetate, fumarate, gluconate, glutamate, hydrabamine, hydrobromine, hydrochloride, iodide, isothionate, lactate, malate, maleate, mandelate, mesylate, nitrate, oleate, pamoate, palmitate, phosphate/diphosphate, salicylate, stearate, sulfate, succinate, tartrate, tosylate.

Certain compounds of the Formula (I) may exist in various stereoisomeric or tautomeric forms and mixtures thereof. The present invention encompasses all such dual α₁ₐ/α_{1d} adrenoceptor inhibiting compounds, including active compounds in the form of essentially pure enantiomers, racemic mixtures, pure geometric isomers (such as cis and trans stereoisomers), mixtures of geometric isomers, and tautomers.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers, or enantiomers).

The term “stereoisomer” refers to isomers of identical constitution that differ in the arrangement of their atoms in space. Enantiomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superimposable. The term “diastereomer” refers to stereoisomers that are not related as mirror images. The symbols “R” and “S” represent the configuration of substituents around a chiral carbon atom(s). The symbols “R*” and “S*” denote the relative configurations of of substituents around a chiral carbon atom(s). Where the compounds of the present application have at least one stereocenter, they accordingly exist as enantiomers. Where the compounds according to the present invention posses two or more stereocenters, they may additionally exist as diastereoisomers. It is to be understood that all such isomers and mixtures thereof are encompassed within the scope to the present invention.

The term “racemate” or “racemic mixture” refers to a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) configuration, the substituents are on opposite sides in relationship to the carbon-carbon double bond; in the “Z” (same sided) configuration, the substituents are oriented on the same side in relationship to the carbon-carbon double bond. Substituent atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

Furthermore, compounds of the present invention may have one or more polymorph or amorphous crystalline forms and as such are intended to be included in the scope of the invention. In addition, some of the compounds may form solvates with water (i.e., hydrates) or common organic solvents, and such are also intended to be encompassed within the scope of this invention.

Chemical Nomenclature and Definitions

As used herein, the following terms are intended to have the following meanings (additional definitions are provided where needed throughout the Specification):

The term “C₁₋₈alkyl” whether used alone or as part of a substituent group, means a straight or branched chain monovalent hydrocarbon alkyl radical or alkyldiyl linking group, wherein the radical is derived by the removal of one hydrogen atom from a single carbon atom and the linking group is derived by the removal of one hydrogen atom from each of two carbon atoms in the chain. Typical alkyl groups comprising from 1 to 8 carbon atoms include, for example methyl, ethyl, 1-propyl, 2-propyl, 1-butyl, 2-butyl, tertiary butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Examples include C₁₋₈alkyl, C₁₋₆alkyl and C₁₋₄alkyl groups and the like.

The term “C₂₋₈alkenyl” whether used alone or as part of a substituent group, means a straight or branched chain monovalent hydrocarbon alkyl radical or alkyldiyl linking group having at least one carbon-carbon double bond, whereby the double bond is derived by the removal of one hydrogen atom from each of two adjacent carbon atoms of the radical or linking group. Atoms may be oriented about the double bond in either the cis or trans conformation. Typical alkenyl groups comprising from 2 to 8 carbon atoms include, for example, ethenyl, propenyl, allyl (2-propenyl), butenyl, pentenyl, hexenyl and the like. Examples include C₂₋₈alkenyl and C₂₋₄alkenyl groups and the like.

The term “C₂₋₈alkynyl” whether used alone or as part of a substituent group, means a straight or branched chain monovalent hydrocarbon alkyl radical or alkyldiyl linking group having at least one carbon-carbon triple bond, whereby the triple bond is derived by the removal of two hydrogen atoms from each of two adjacent carbon atoms of the radical or linking group. Typical alkynyl groups comprising from 2 to 8 carbon atoms include, for example, ethynyl, propynyl, butynyl, pentynyl, hexynyl and the like. Examples include C₂₋₈alkynyl and C₂₋₄alkynyl groups and the like.

The term “C₁₋₈alkoxy” whether used alone or as part of a substituent group, refers to an alkyl or alkyldiyl radical attached through an oxygen linking atom. Typical alkoxy groups comprising from 1 to 8 carbon atoms include, for example, methoxy, ethoxy, propoxy, butoxy, pentoxy, hexoxy, heptoxy, octoxy and the like. Examples include C₁₋₈alkoxy or C₁₋₄alkoxy groups and the like.

The term “C₃₋₁₂cycloalkyl” whether used alone or as part of a substituent group, refers to a saturated or partially unsaturated, monocyclic or polycyclic hydrocarbon ring system radical derived by the removal of one hydrogen atom from a single ring carbon atom. Typical cycloalkyl radicals include cyclopropyl, cyclobutyl, cyclopentyl, cyclohexyl, cycloheptyl, cyclooctyl, 1H-indenyl, indanyl, tetrahydro-naphthalenyl and the like. Examples include C₃₋₈cycloalkyl, C₅₋₈cycloalkyl or C₃₋₁₀cycloalkyl groups and the like.

The term “heterocyclyl” whether used alone or as part of a substituent group, refers to a saturated or partially unsaturated monocyclic or polycyclic ring radical derived by the removal of one hydrogen atom from a single carbon or nitrogen ring atom. Typical heterocyclyl radicals include 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furyl, tetrahydro-thienyl, tetrahydro-pyranyl, tetrahydro-pyridazinyl, 1,3-benzodioxolyl (also referred to as benzo[1,3]dioxolyl) or 2,3-dihydro-1,4-benzodioxinyl (also referred to as 2,3-dihydro-benzo[1,4]dioxinyl) and the like.

The term “hetero” used as a prefix for a ring system refers to the replacement of at least one ring carbon atom with one or more heteroatoms independently selected from N, S, O or P. Examples include rings wherein 1, 2, 3 or 4 ring members are a nitrogen atom; or, 0, 1, 2 or 3 ring members are nitrogen atoms and 1 member is an oxygen or sulfur atom. When allowed by available valences, up to two adjacent ring members may be heteroatoms; wherein one heteroatom is nitrogen and the other is one heteroatom selected from N, S or O.

The term “aryl,” whether used alone or as part of a substituent group, refers to an aromatic monocyclic or polycyclic hydrocarbon ring radical derived by the removal of one hydrogen atom from a single carbon atom of the ring system. Typical aryl radicals include phenyl, naphthalenyl, fluorenyl, indenyl, azulenyl, anthracenyl and the like.

The term “aromatic” refers to a cycloalkylic hydrocarbon ring system having an unsaturated, conjugated n electron system.

The term “heteroaryl,” whether used alone or as part of a substituent group, refers to an heteroaromatic monocyclic or polycyclic hydrocarbon ring radical derived by the removal of one hydrogen atom from a single ring carbon atom of the ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, azaindolyl, isoindolyl, benzo[b]furyl, benzo[b]thienyl, indazolyl, azaindazolyl, benzimidazolyl, benzthiazolyl, benzoxazolyl, benzisoxazolyl, benzothiadiazolyl, benzotriazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “halogen” includes fluoro, chloro, bromo, and iodo.

The term “substituted,” refers to a core molecule on which one or more hydrogen atoms have been replaced with one or more functional radical moieties. The number that is allowed by available valences limits the amount of substituents. Substitution is not limited to the core molecule, but may also occur on a substituent radical, whereby the substituent radical becomes a linking group.

The term “independently selected” refers to one or more substituents selected from a group of substituents variable group, wherein the selected substituents may be the same or different.

The term “dependently selected” refers to one or more substituents specified in an indicated combination of structure variables.

Therapeutic Use

In an example of therapeutic use, the compounds of the present invention are modulators for the α₁ₐ-AR and α_{1d}-AR subtypes and are useful for the treatment of BPH and/or LUTS.

In another example of therapeutic use, the modulator compounds are agonists, inverse-agonists or antagonists for each of the α₁ₐ-AR and α_{1d}-AR subtypes. In another example, the modulator compounds are selective antagonists for each of the α₁ₐ-AR and α_{1d}-AR subtypes.

In another example of therapeutic use, the modulator compounds are agonists, inverse-agonists or antagonists for both the α₁ₐ-AR and α_{1d}-AR subtypes. In another example, the modulator compounds are selective agonists for both the α₁ₐ-AR and α_{1d}-AR subtypes. In another example, the modulator compounds are selective inverse-agonists for both the α₁ₐ-AR and α_{1d}-AR subtypes. In another example, the modulator compounds are selective antagonists for both the α₁ₐ-AR and α_{1d}-AR subtypes.

The binding affinities for instant compounds demonstrate selectivity as modulators for either the α₁ₐ-AR or α_{1d}-AR subtype when compared to the binding affinities for other types of α₁-ARs.

Furthermore, the binding affinities for instant compounds demonstrate selectivity as modulators for both the α₁ₐ-AR and α_{1d}-AR subtypes when compared to the binding affinities for other types of α₁-ARs.

Accordingly, the modulator compounds of the present invention are useful for treating, ameliorating or preventing a plurality of α₁ₐ-AR and α_{1d}-AR mediated disorders or diseases. The usefulness of a compound of the present invention or pharmaceutical composition thereof as an α₁ₐ-AR or α_{1d}-AR modulator or as a dual α₁ₐ and α_{1d}-AR modulator can be determined according to the methods disclosed herein.

The term “α₁ₐ-AR and α_{1d}-AR mediated disorder or disease” means disorders or diseases such as, but not limited to, contractions of the prostate, bladder and other organs of the lower urinary tract with or without an effect on blood pressure. The scope of such use includes the treatment of BPH and/or LUTS.

The term “LUTS” means disorders or diseases such as, but not limited to, filling symptoms, urgency, incontinence and nocturia, as well as voiding problems such as weak stream, hesitancy, intermittency, incomplete bladder emptying and abdominal straining.

The present invention thereby includes a method for treating, ameliorating or preventing an α₁ₐ-AR and α_{1d}-AR mediated disorder or disease in a patient in need of such treatment comprising administering to the patient an effective amount of a compound of Formula (I) or pharmaceutical composition thereof.

The present invention thereby includes a method for treating, ameliorating or preventing BPH and/or LUTS in a patient in need of such treatment comprising administering to the patient an effective amount of a compound of Formula (I) or pharmaceutical composition thereof.

The term “patient” means an animal, preferably a mammal, most preferably a human, which has been the object of treatment, prevention, observation or experiment.

The term “administering” is to be interpreted liberally in accordance with the methods of the present invention. Such methods include therapeutically or prophylactically administering an effective amount of a composition or medicament of the present invention at different times during the course of a therapy or concurrently in a combination form. Prophylactic administration can occur prior to the manifestation of symptoms characteristic of an α₁ₐ and/or α_{1d} adrenoreceptor mediated disorder or disease such that the disorder or disease is treated, ameliorated, prevented or otherwise delayed in its progression. The methods of the present invention are further to be understood as embracing all therapeutic or prophylactic treatment regimens used by those skilled in the art.

The term “effective amount” refers to that amount of active compound or pharmaceutical agent that elicits the biological or medicinal response in a tissue system, animal or human, that is being sought by a researcher, veterinarian, medical doctor, or other clinician, which includes treating, ameliorating or preventing the symptoms of a syndrome, disorder or disease being treated.

In an example of the method for treating, ameliorating or preventing an α₁ₐ-AR and α_{1d}-AR mediated disorder or disease described herein, the method includes treating a patient suffering from BPH and/or LUTS comprising administering to the patient an effective amount of a combination product comprising a compound of Formula (I) or pharmaceutical composition thereof in combination with a BPH and/or LUTS therapeutic agent.

The BPH and/or LUTS therapeutic agent includes a human testosterone 5α-reductase inhibitor agent or 5-αreductase isoenzyme 2 inhibitor agent (such as finasteride or durasteride and the like or mixtures thereof), a NK-1 inhibitor, an anti-androgen receptor agonist, an androgen receptor antagonist, a selective androgen receptor modulators, a PDE inhibitor, a urinary incontinence drugs (e.g. anti-muscarinics) or a 5HT-receptor modulator.

With regard to the method for administering a combination product, the term “effective amount” means that amount of the compound of Formula (I) or pharmaceutical composition thereof in combination with that amount of the therapeutic agent, which have been adjusted to treat, ameliorate or prevent the symptoms of a syndrome, disorder or disease being treated.

As those skilled in the art will appreciate, the dosages of the compound of Formula (I) or pharmaceutical composition thereof and the therapeutic agent may be independently optimized and combined to achieve a synergistic result wherein the pathology is reduced more than it would be if either agent were used alone. In accordance with the method of the present invention, the individual components of the combination can be administered separately at different times during the course of therapy or concurrently in divided or single combination forms. The instant invention is therefore to be understood as embracing all such regimes of simultaneous or alternating treatment and the term “administering” is to be interpreted accordingly.

In solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogenous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. An enteric layer can separate the two components. That enteric layer serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

As used herein, the term “composition” is intended to encompass a product comprising the specified ingredients in the specified amounts, as well as any product which results, directly or indirectly, from combination of the specified ingredients in the specified amounts.

An effective but non-toxic amount of the compound desired can be employed as a α₁ₐ/α_{1d} antagonistic agent. Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium and elimination of a drug.

Compounds of Formula (I) may be administered in any of the foregoing compositions and according to dosage regimens established in the art whenever inhibition of the human α₁ₐ-AR or α₁ₐ-AR is required. Such inhibition includes inhibition of the human α₁ₐ-AR or α_{1d}-AR, selective inhibition of the human α₁ₐ-AR or α_{1d}-AR, dual inhibition of the human α₁ₐ-AR and α_{1d}-AR or selective, dual inhibition of the human α₁ₐ-AR and α_{1d}-AR. The compounds of Formula (I) may be used alone at appropriate dosages defined by routine testing in order to obtain optimal antagonism of the human α₁ₐ-AR or α_{1d}-AR while minimizing any potential toxicity.

The daily dosage of the products may be varied over a wide range from about 0.001 to about 3,000 mg per adult human per day. For oral administration, the compositions are preferably provided in the form of tablets containing 0.01, 0.05, 0.1, 0.5, 1.0, 2.5, 5.0, 10.0, 15.0, 25.0, 50.0 and milligrams of the active ingredient for the symptomatic adjustment of the dosage to the patient to be treated. A medicament typically contains from about 0.01 mg to about 500 mg of the active ingredient, preferably, from about 0.01 mg to about 3000 mg of active ingredient.

An effective amount of a compound of Formula (I) is a dosage level range of from about 0.0002 mg/kg to about 20 mg/kg of body weight per day. Preferably, the range is from about 0.001 to 10 mg/kg of body weight per day. More preferably, the range is from about 0.001 mg/kg to 7 mg/kg of body weight per day. The compounds may be administered on a regimen of 1 to 4 times per day.

When compounds of Formula (I) are administered in a combination product, the compound of Formula (I) or pharmaceutical composition thereof and the therapeutic agent may be co-administered or sequentially administered whereby the effects of BPH and/or LUTS is treated, ameliorated or prevented.

The effective amount of the therapeutic agent selected from a human testosterone 5α-reductase inhibitor agent or 5-α reductase isoenzyme 2 inhibitor agent (such as finasteride or durasteride and the like or mixtures thereof), a NK-1 inhibitor, an anti-androgen receptor agonist, an androgen receptor antagonist, a selective androgen receptor modulators, a PDE inhibitor, a urinary incontinence drugs (e.g. anti-muscarinics) or a 5HT-receptor modulator is a dosage level range of from about 0.0002 mg/kg to about 20 mg/kg of body weight per day. Preferably, the range is from about 0.001 to 10 mg/kg of body weight per day. More preferably, the range is from about 0.001 mg/kg to 7 mg/kg of body weight per day.

In one example of the combination product, the therapeutic agent is finasteride. The method for administering a combination product further comprises administering to the patient an effective amount of a compound of Formula (I) or pharmaceutical composition thereof in combination with finasteride.

The effective amount of finasteride administered in such a combination product is a dosage level range of from about 0.01 mg per day to about 50 mg per day. Preferably, the range is from about 0.2 mg per day to about 10 mg per day. More preferably, the range is from about 1 mg per day to about 7 mg per day. Most preferably, the dosage level is about 5 mg per day.

In yet another aspect, the present invention provides diagnostic compositions which are used for in vivo imaging of a α₁ₐ and α_{1d} adrenoreceptors, comprising a compound of the present invention which is capable of being detected outside the body. Preferred are compositions comprising a compound of the present invention and a detectable label, such as a radioactive atom.

In yet another aspect the present invention provides compounds which are useful as ligands for use in assays relating to a α₁ₐ/α_{1d} adrenoreceptors.

Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3rd Edition, John Wiley & Sons, 1999. The protecting groups may be removed at a convenient subsequent stage using methods known in the art.

Synthetic Routes

Where the processes for the preparation of the compounds according to the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

The terms used in describing the invention are commonly used and known to those skilled in the art. Some reagents are referred to as a chemical formula. Other reagents are referred to as abbreviations known to persons skilled in the art. When used herein, the following abbreviations have the indicated meanings:

Cpd compound

DCM dichloromethane

min/hr(s)/d(s) minute/hour(s)/day(s)

M.P. melting point in ° C.

MS Mass Spectrum in m/z (M+H⁺)

RT/rt/r.t. room temperature

TEA triethylamine

THF tetrahydrofuran

Specific compounds which are representative of the invention may be prepared as per the following examples offered by way of illustration and not by way of limitation. No attempt has been made to optimize the yields obtained in any of the reactions. One skilled in the art would know how to increase such yields through routine variations in reaction times, temperatures, solvents and/or reagents. Additional compounds may be made according to the synthetic methods of the present invention by one skilled in the art, differing only in possible starting materials, reagents and conditions used in the instant methods.

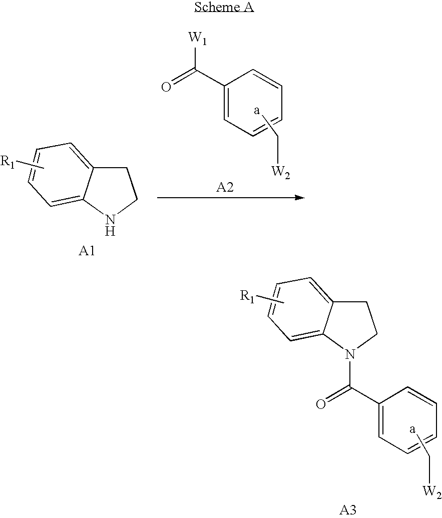

A solution of a substituted indole Compound A1 in DCM and TEA is cooled to about −60° C. A solution of a substituted Compound A2 (wherein W₁ is a an appropriate leaving group, such as, for example, halo, more specifically chloride and wherein W₂ is an appropriate leaving group, such as, for example, halo, more specifically chloro) in DCM (wherein “a” represents a point of attachment on the phenyl ring of Compound A2 for the methylene-W₂ substituent) is then added dropwise to the solution of Compound A1. The reaction mixture is warmed to RT and stirred for about 5 hrs. The mixture is poured into cold water and the organic layer is separated, washed (preferably with brine), dried (preferably with Na₂SO₄) and concentrated in vacuo to give the methanone Compound A3.

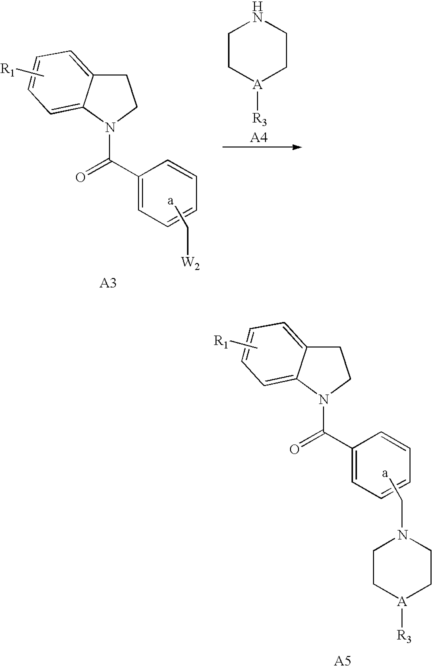

Compound A3 (wherein is W₂ is an appropriate leaving group, such as, for example, halo, more specifically, chloro) is treated with a solution of a substituted heterocyclyl Compound A4 and potassium carbonate in acetonitrile. The reaction mixture is refluxed for about 4 hrs, then filtered, cooled and concentrated in vacuo. The residue is treated with water and extracted as needed (preferably twice) with DCM. The combined organic layers are washed (preferably with brine), dried (preferably with Na₂SO₄) and concentrated in vacuo to give a Compound A5 of Formula (I).

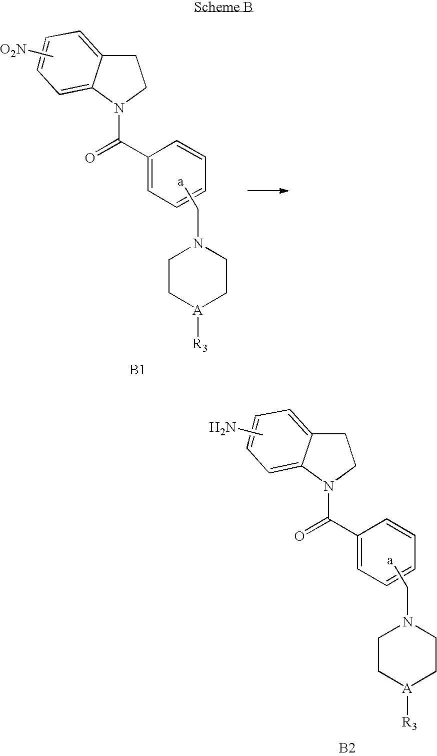

When R₁ is nitro, as represented by Compound B1, the nitro substituent can be converted to a primary amine Compound B2 by treating a solution of Compound B1 in a mixture of THF:ethanol with hydrogen gas in the presence of a catalyst (10% Pd/C). The reaction mixture is shaken for about 2.5 hrs on a Parr apparatus, then filtered and concentrated in vacuo to give a solid which is purified via column chromatography (eluted preferably with a 1:1:1 hexane:acetone:chloroform mixture and the like) to give the amine Compound B2.

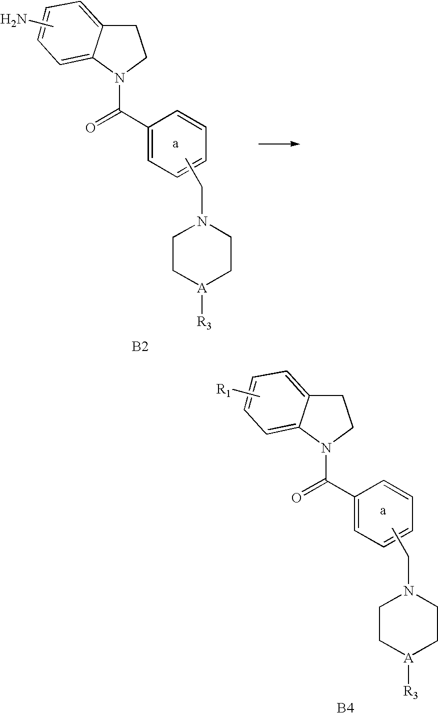

To provide additional compounds representative of the scope of the present invention, a solution of Compound B2 in DCM is further transformed into compounds B4, for instance by reductive alkylation, such can be done by treating with a solution of an appropriate aldehyde in glacial acetic acid and sodium triacetoxyborohydride to provide the reaction product Compound B4. Other transformations are also possible, such as, acylation and sulfonylation. These transformations can be performed according to art known techniques.

Examples of the present invention include compounds of Formula (I), wherein the R₁ substituent can be other than a secondary or tertiary amine, which may be prepared by one skilled in the art substituting the appropriate starting materials, reagents and solvents.

### EXAMPLE 1

### (5-amino-2,3-dihydro-indol-1-yl)-{3-[4-(2-isopropoxy-phenyl)-piperazin-1-ylmethyl]-phenyl}-methanone (Cpd 1)

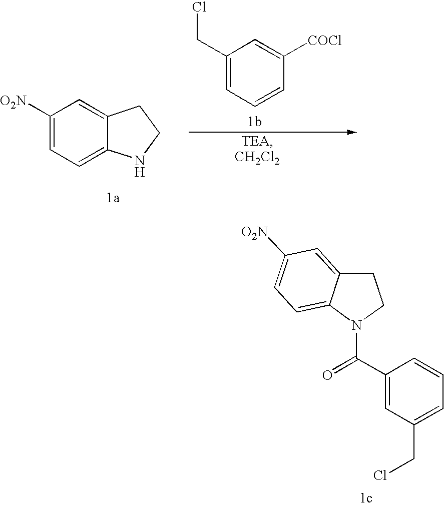 A solution of 3-chloromethyl-benzoyl chloride Compound 1b (11.5 g, 0.06 mol) in DCM (25 mL) was added dropwise to a cooled (−60° C.) solution of 5-nitro-2,3-dihydro-1H-indole Compound 1a (10 g, 0.06 mol) in DCM (200 mL) and TEA (10 mL, 0.07 mol). The reaction mixture was warmed to room temperature and stirred for 5 hrs. The mixture was poured into cold water and the organic layer was separated, washed with brine, dried (Na₂SO₄) and concentrated in vacuo to give the (3-chloromethyl-phenyl)-(5-nitro-2,3-dihydro-indol-1-yl)-methanone Compound 1c (20.69 g, >100%) as a light yellow solid which contained residual DCM.

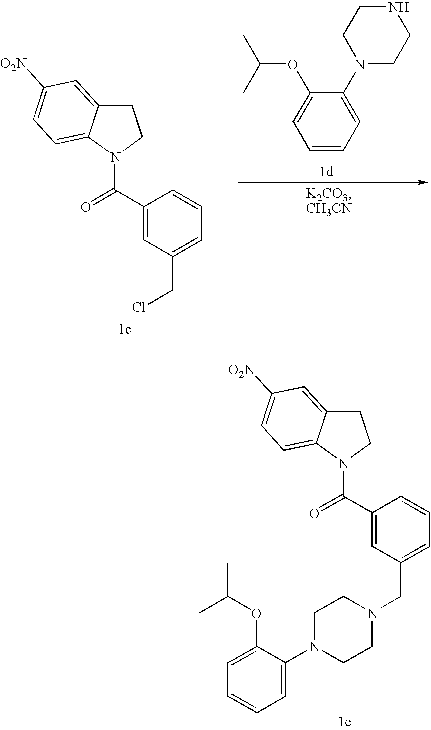

Compound 1c (11.75 g, 0.037 mol) was treated with 1-(2-isopropoxy-phenyl)-piperazine monofumarate Compound 1d (12.46 g, 0.037 mol) and potassium carbonate (15.26 g, 0.11 mol) in acetonitrile (240 mL) and the resulting mixture was refluxed for 4 hrs. The reaction mixture was filtered, cooled and evaporated in vacuo to give a syrup which was treated with water and extracted twice with DCM. The combined organic layers were washed with brine, dried (Na₂SO₄) and concentrated in vacuo to give {3-[4-(2-isopropoxy-phenyl)-piperazin-1-ylmethyl]-phenyl}-(5-nitro-2,3-dihydro-indol-1-yl)-methanone Compound 1e (20.26 g, >100%) as a light yellow solid which contained residual DCM.

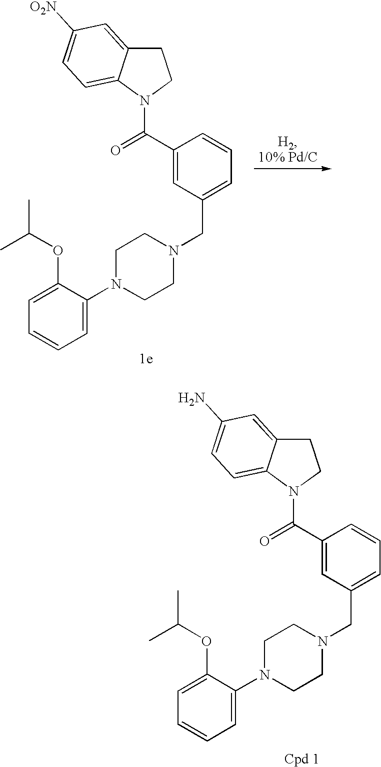

Compound 1e (6.1 g, 0.01 mol) in a mixture of THF:ethanol (50:100 mL) was treated with a catalyst (10% Pd/C, 0.60 g) and shaken for 2.5 h on a Parr apparatus under a hydrogen atmosphere, filtered and concentrated in vacuo to give a yellow solid which was purified via column chromatography (1:1:1 hexane:acetone:chloroform) to give Compound 1 (3.47 g, 74%) as a deep yellow solid. MS, m/z 471 (M+H); M.P. 137-139° C.

Following the procedure of Example 1, up until the preparation of intermediate 1e, substituting the appropriate starting materials, reagents and solvents, the following compounds were prepared:

| Cpd | Name | MS |
|---|---|---|
| 30 | (2,3-dihydro-indol-1-yl)-{3-[4-(2-methoxy-phenyl)-piperazin-1-ylmethyl]- | 428 |
| | phenyl}-methanone | |
| 31 | (2,3-dihydro-indol-1-yl)-{3-[4-(2-cyano-phenyl)-piperazin-1-ylmethyl]- | 423 |
| | phenyl}-methanone | |
| 54 | 4-[3-(2,3-dihydro-indole-1-carbonyl)-benzyl]-piperazine-1-carboxylic acid | 422 |
| | tert-butyl ester | |
| 56 | (2,3-dihydro-indol-1-yl)-{3-[4-(2-isopropoxy-benzyl)-piperazin-1- | 470 |
| | ylmethyl]-phenyl}-methanone | |
| 63 | (2,3-dihydro-indol-1-yl)-(3-{4-[2-(2,2,2-trifluoro-ethoxy)-phenyl]- | 496 |
| | piperazin-1-ylmethyl}-phenyl)-methanone | |
| 64 | (2,3-dihydro-indol-1-yl)-{3-[4-(2-methoxy-benzyl)-piperazin-1-ylmethyl]- | 442 |
| | phenyl}-methanone | |
| 65 | (2,3-dihydro-indol-1-yl)-{3-[4-(2-isobutoxy-phenyl)-piperazin-1-ylmethyl]- | 470 |
| | phenyl}-methanone | |
| 66 | (5-bromo-2,3-dihydro-indol-1-yl)-{3-[4-(2-isopropoxy-phenyl)-piperazin-1- | 534 |
| | ylmethyl]-phenyl}-methanone | |
| 67 | (5-chloro-2,3-dihydro-indol-1-yl)-{3-[4-(2-isopropoxy-phenyl)-piperazin-1- | 490 |
| | ylmethyl]-phenyl}-methanone | |
| 68 | (5-fluoro-2,3-dihydro-indol-1-yl)-{3-[4-(2-isopropoxy-phenyl)-piperazin-1- | 474 |
| | ylmethyl]-phenyl}-methanone | |
| 69 | (2,3-dihydro-indol-1-yl)-{3-[4-(2-ethoxy-phenyl)-piperazin-1-ylmethyl]- | 442 |
| | phenyl}-methanone | |
| 70 | 1-{3-[4-(2-isopropoxy-phenyl)-piperazin-1-ylmethyl]-benzoyl}-2,3- | 563 |
| | dihydro-1H-indole-5-sulfonic acid dimethylamide | |
| 71 | {3-[4-(2-isopropoxy-phenyl)-piperazin-1-ylmethyl]-phenyl}-(5- | 534 |
| | methanesulfonyl-2,3-dihydro-indol-1-yl)-methanone | |
| 72 | {3-[4-(2-isopropoxy-phenyl)-piperazin-1-ylmethyl]-phenyl}-(5-phenyl-2,3- | 532 |
| | dihydro-indol-1-yl)-methanone | |

### EXAMPLE 2

### {3-[4-(2-isopropoxy-phenyl)-piperazin-1-ylmethyl]-phenyl}-[5-(4-trifluoromethyl-benzylamino)-2,3-dihydro-indol-1-yl]-methanone (Cpd 8)

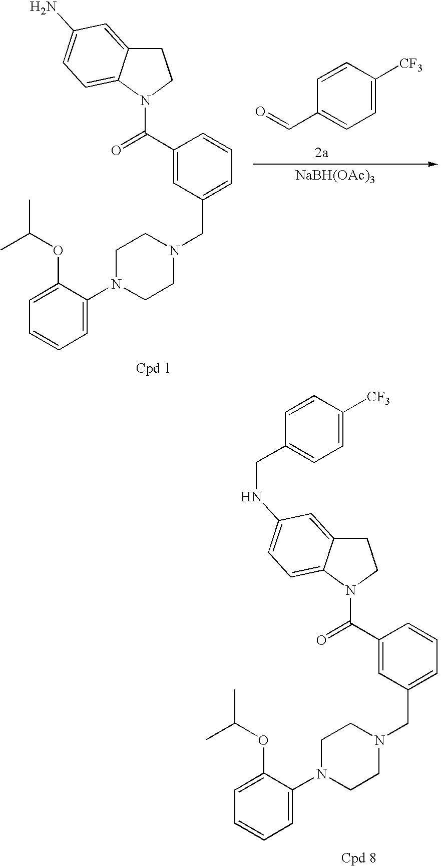

A solution of (5-amino-2,3-dihydro-indol-1-yl)-{3-[4-(2-isopropoxy-phenyl)-piperazin-1-ylmethyl]-phenyl}-methanone Compound 1 (0.15 g, 0.3 mmol) in DCM (4 mL) was treated with 4-trifluoromethyl-benzaldehyde Compound 2a (0.06 g, 0.3 mmol), glacial acetic acid (0.02 mL) and sodium triacetoxyborohydride (0.15 g, 0.7 mmol). The mixture was stirred under an inert atmosphere for 24 hrs, then treated with 1N NaOH (8 mL) and stirred for 60 min. The layers were separated. The organic layer was dried (Na₂SO₄), filtered and concentrated in vacuo to give Compound 8 (0.16 g) as an off white solid. MS m/z 629 (M+H⁺); M.P. 74-76° C.

Following the procedure of Example 2, substituting the appropriate starting materials, reagents and solvents, the following compounds were prepared:

| Cpd | Name | MS | M.P. |
|---|---|---|---|
| 9 | [5-(2,6-difluoro-benzylamino)-2,3-dihydro-indol-1-yl]-{3-[4-(2- | 597 | 127-130 |
| | isopropoxy-phenyl)-piperazin-1-ylmethyl]-phenyl}-methanone | | |
| 23 | {3-[4-(2-isopropoxy-phenyl)-piperazin-1-ylmethyl]-phenyl}-[5-(4- | 673 | 116-118 |
| | octyl-benzylamino)-2,3-dihydro-indol-1-yl]-methanone | | |
| 24 | [5-(4-diethoxymethyl-benzylamino)-2,3-dihydro-indol-1-yl]-{3-[4- | 663 | — |
| | (2-isopropoxy-phenyl)-piperazin-1-ylmethyl]-phenyl}-methanone | | |
| 25 | [5-(2,6-dimethoxy-benzylamino)-2,3-dihydro-indol-1-yl]-{3-[4-(2- | 621 | 79-81 |
| | isopropoxy-phenyl)-piperazin-1-ylmethyl]-phenyl}-methanone | | |
| 27 | {5-[(benzo[1,3]dioxol-5-ylmethyl)-amino]-2,3-dihydro-indol-1- | 605 | — |
| | yl}-{3-[4-(2-isopropoxy-phenyl)-piperazin-1-ylmethyl]-phenyl}- | | |
| | methanone | | |

### EXAMPLE 3

### (6-amino-2,3-dihydro-indol-1-yl)-{3-[4-(2-isopropoxy-phenyl)-piperazin-1-ylmethyl]-phenyl}-methanone (Cpd 5) [6-(2,6-difluoro-benzylamino)-2,3-dihydro-indol-1-yl]-{3-[4-(2-isopropoxy-phenyl)-piperazin-1-ylmethyl]-phenyl}-methanone (Cpd 6)

Using the procedure of Example 1,6-nitro-2,3-dihydro-1H-indole Compound 3a was used in place of 5-nitro-2,3-dihydro-1H-indole Compound 1a to provide Compound 5.

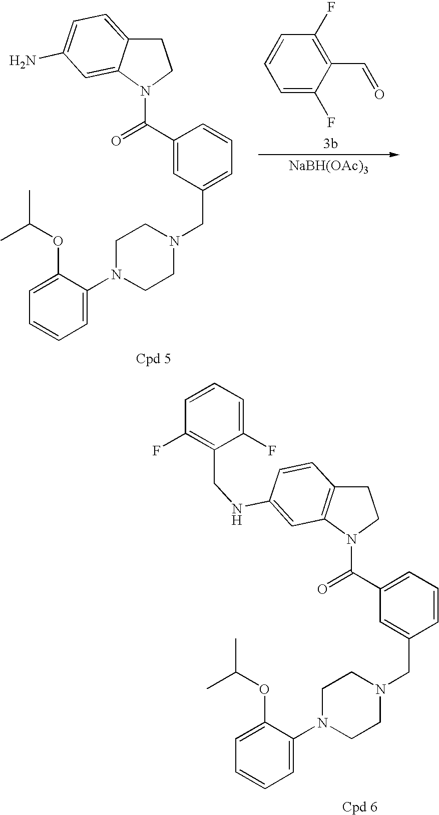

Using the procedure of Example 2, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 and 2,6-difluoro-benzaldehyde Compound 3b was used in place of 4-trifluoromethyl-benzaldehyde Compound 2a to provide Compound 6. MS m/z 597 (M+H⁺); M.P. 137-139° C.

Following the procedure of Example 3, substituting the appropriate starting materials, reagents and solvents, the following compounds were prepared:

| Cpd | Name | MS | M.P. |
|---|---|---|---|
| 7 | [6-(2,6-dimethoxy-benzylamino)-2,3-dihydro-indol-1-yl]-{3-[4- | 621 | — |
| | (2-isopropoxy-phenyl)-piperazin-1-ylmethyl]-phenyl}- | | |
| | methanone | | |
| 10 | {3-[4-(2-isopropoxy-phenyl)-piperazin-1-ylmethyl]-phenyl}-[6- | 673 | — |
| | (4-octyl-benzylamino)-2,3-dihydro-indol-1-yl]-methanone | | |
| 11 | [6-(2,6-dimethoxy-benzylamino)-2,3-dihydro-indol-1-yl]-{3-[4- | 663 | 156-160 |
| | (2-isopropoxy-phenyl)-piperazin-1-ylmethyl]-phenyl}- | | |
| | methanone | | |
| 41 | {6-[(benzo[1,3]dioxol-5-ylmethyl)-amino]-2,3-dihydro-indol-1- | 605 | — |
| | yl}-{3-[4-(2-isopropoxy-phenyl)-piperazin-1-ylmethyl]-phenyl}- | | |
| | methanone | | |
| 44 | [6-(2-fluoro-benzylamino)-2,3-dihydro-indol-1-yl]-{3-[4-(2- | 579 | — |
| | isopropoxy-phenyl)-piperazin-1-ylmethyl]-phenyl}-methanone | | |

### EXAMPLE 4

### naphthalene-2-sulfonic acid (1-{3-[4-(2-isopropoxy-phenyl)-piperazin-1-ylmethyl]-benzoyl}-2,3-dihydro-1H-indol-5-yl)-amide (Cpd 2)

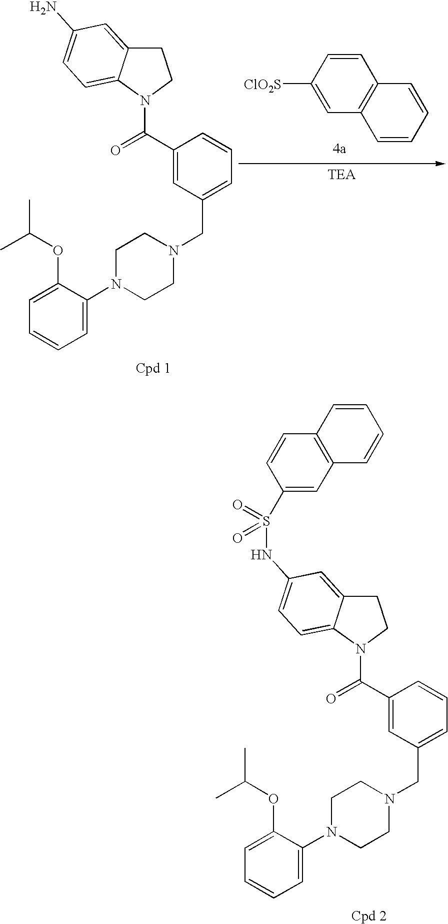

A solution of (5-amino-2,3-dihydro-indol-1-yl)-{3-[4-(2-isopropoxy-phenyl)-piperazin-1-ylmethyl]-phenyl}-methanone Compound 1 (0.15 g, 0.32 mmol) in DCM (5 mL) was treated with TEA (0.038 g, 0.37 mmol) and naphthalene-2-sulfonyl chloride Compound 4a (0.08 g, 0.35 mmol). The mixture was stirred for 8 hrs and then treated with water. The organic layer was separated, dried (Na₂SO₄), filtered and concentrated in vacuo to give a residue which was purified via column chromatography (2:1:1 hexane:acetone:chloroform) to give Compound 2 (0.125 g) as a yellow solid. MS m/z 661 (M+H); M.P. 73-76° C.

Following the procedure of Example 4, substituting the appropriate starting materials, reagents and solvents, the following compounds were prepared:

| Cpd | Name | MS | M.P. |
|---|---|---|---|
| 3 | 5-dimethylamino-naphthalene-1-sulfonic | 704 | 80-83 |
| | acid (1-{3-[4-(2-isopropoxy-phenyl)- | | |
| | piperazin-1-ylmethyl]-benzoyl}-2,3-dihydro- | | |
| | 1H-indol-5-yl)-amide | | |
| 19 | N-[4-(1-{3-[4-(2-isopropoxy-phenyl)-piperazin- | 668 | 132-134 |
| | 1-ylmethyl]-benzoyl}-2,3-dihydro-1H- | | |
| | indol-5-ylsulfamoyl)-phenyl]-acetamide | | |

### EXAMPLE 5

### 5-dimethylamino-naphthalene-1-sulfonic acid (1-{3-[4-(2-isopropoxy-phenyl)-piperazin-1-ylmethyl]-benzoyl}-2,3-dihydro-1H-indol-6-yl)-amide (Cpd 32)

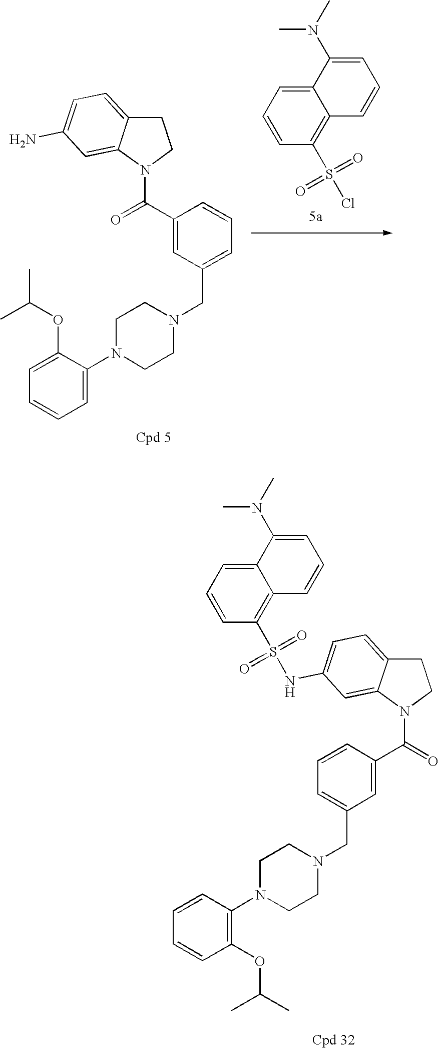

Using the procedure of Example 4, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 and 5-dimethylamino-naphthalene-1-sulfonyl chloride Compound 5a was used in place of naphthalene-2-sulfonyl chloride Compound 4a to provide Compound 32. MS m/z 704 (M+H⁺); M.P. 137-139° C.

Following the procedure of Examples 4 and 5, substituting the appropriate starting materials, reagents and solvents, the following compounds were prepared:

| Cpd | Name | MS | M.P. |
|---|---|---|---|
| 15 | N-[4-(1-{3-[4-(2-isopropoxy-phenyl)-piperazin-1-ylmethyl]- | 668 | — |
| | benzoyl}-2,3-dihydro-1H-indol-6-ylsulfamoyl)-phenyl]- | | |
| | acetamide | | |
| 18 | 5-chloro-3-methyl-benzo[b]thiophene-2-sulfonic acid (1-{3-[4- | 716 | 192-195 |
| | (2-isopropoxy-phenyl)-piperazin-1-ylmethyl]-benzoyl}-2,3- | | |
| | dihydro-1H-indol-6-yl)-amide | | |
| 45 | 2,6-difluoro-N-(1-{3-[4-(2-isopropoxy-phenyl)-piperazin-1- | 647 | — |
| | ylmethyl]-benzoyl}-2,3-dihydro-1H-indol-6-yl)- | | |
| | benzenesulfonamide | | |
| 46 | N-(1-{3-[4-(2-isopropoxy-phenyl)-piperazin-1-ylmethyl]- | 625 | — |
| | benzoyl}-2,3-dihydro-1H-indol-6-yl)-4-methyl- | | |
| | benzenesulfonamide | | |
| 47 | ethanesulfonic acid (1-{3-[4-(2-isopropoxy-phenyl)-piperazin-1- | 563 | — |
| | ylmethyl]-benzoyl}-2,3-dihydro-1H-indol-6-yl)-amide | | |

### EXAMPLE 6

### 1-(1-{3-[4-(2-isopropoxy-phenyl)-piperazin-1-ylmethyl]-benzoyl}-2,3-dihydro-1H-indol-5-yl)-3-phenyl-urea (Cpd 21)

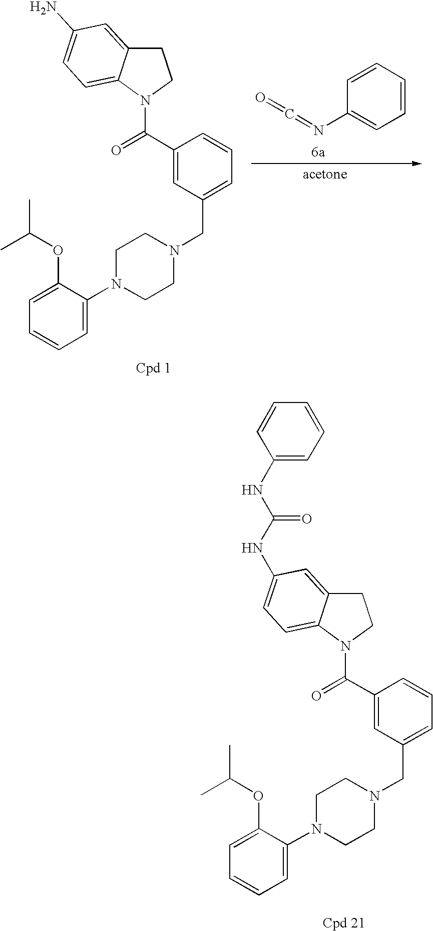

A solution of (5-amino-2,3-dihydro-indol-1-yl)-{3-[4-(2-isopropoxy-phenyl)-piperazin-1-ylmethyl]-phenyl}-methanone Compound 1 (0.15 g, 0.32 mmol) in acetone (5 mL) was treated with isocyanato-benzene Compound 6a (0.038 g, 0.32 mmol). The reaction mixture was stirred at rt for 4 hrs, then concentrated in vacuo to a residue which was purified on a column (30:30:30:5 hexane:chloroform:acetone:methanol) to give Compound 21 as a colorless syrup (0.08 g). MS m/z 590 (M+H).

Following the procedure of Example 6, substituting the appropriate starting materials, reagents and solvents, the following compounds were prepared:

| Cpd | Name | MS |
|---|---|---|
| 14 | 1-(2,4-dichloro-phenyl)-3-(1-{3-[4-(2- | 659 |
| | isopropoxy-phenyl)-piperazin-1- | |
| | ylmethyl]-benzoyl}- | |
| | 2,3-dihydro-1H-indol-5-yl)-urea | |

### EXAMPLE 7

### 1-(1-{3-[4-(2-isopropoxy-phenyl)-piperazin-1-ylmethyl]-benzoyl}-2,3-dihydro-1H-indol-6-yl)-3-phenyl-urea (Cpd 22)

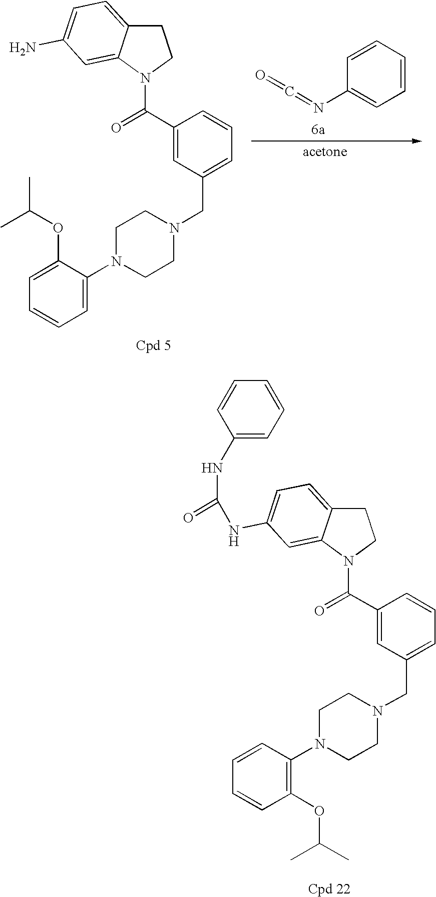

Using the procedure of Example 6, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 to provide Compound 22. MS m/z 590 (M+H⁺).

Following the procedure of Examples 6 and 7, substituting the appropriate starting materials, reagents and solvents, the following compounds were prepared:

| Cpd | Name | MS |
|---|---|---|
| 43 | 3-(2,6-difluoro-phenyl)-1-(1-{3-[4-(2-isopropoxy-phenyl)- | 781 |
| | piperazin-1-ylmethyl]-benzoyl}-2,3-dihydro-1H-indol-6-yl)- | |
| | 1-(2,6-difluorophenylaminocarbonyl)-urea | |
| 49 | (1-{3-[4-(2-isopropoxy-phenyl)-piperazin-1-ylmethyl]- | 514 |
| | benzoyl}-2,3-dihydro-1H-indol-6-yl)-urea | |
| 73 | 1-(2,6-difluoro-phenyl)-3-(1-{3-[4-(2-isopropoxy-phenyl)- | 611 |
| | piperazin-1-ylmethyl]-benzoyl}-2,3-dihydro-1H-indol- | |
| | 6-yl)-urea | |

### EXAMPLE 8

### 1-(1-{3-[4-(2-isopropoxy-phenyl)-piperazin-1-ylmethyl]-benzoyl}-2,3-dihydro-1H-indol-6-yl)-3-phenyl-thiourea (Cpd 13)

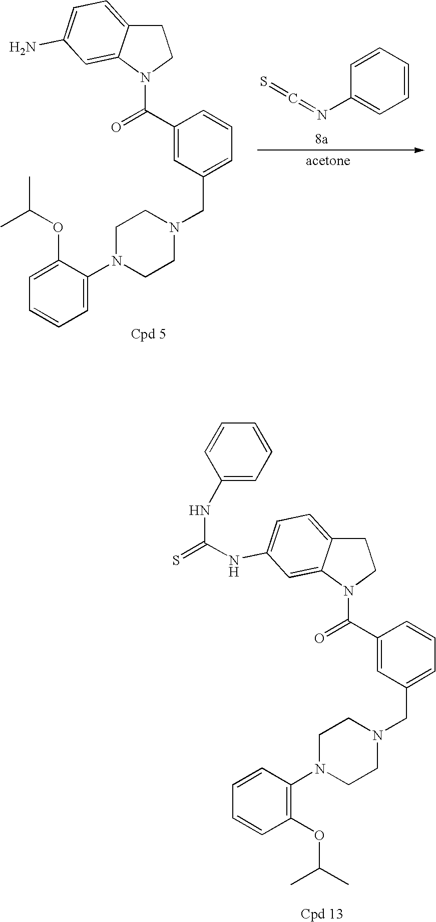

Using the procedure of Example 6, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 and isothiocyanato-benzene Compound 8a was used in place of isocyanato-benzene Compound 6a to provide Compound 13. MS m/z 605 (M+H⁺).

### EXAMPLE 9

### (1-{3-[4-(2-isopropoxy-phenyl)-piperazin-1-ylmethyl]-benzoyl}2,3-dihydro-1H-indol-5-yl)-carbamic acid p-tolyl ester (Cpd 4)

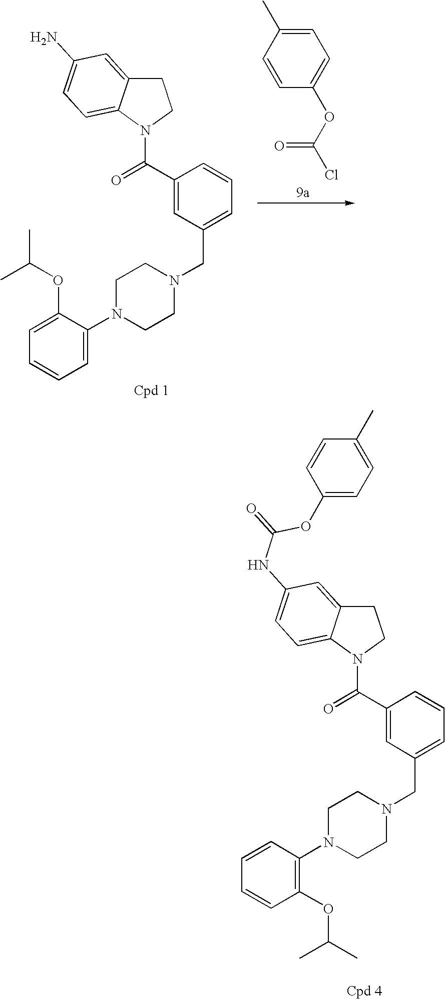

A solution of (5-amino-2,3-dihydro-indol-1-yl)-{3-[4-(2-isopropoxy-phenyl)-piperazin-1-ylmethyl]-phenyl}-methanone Compound 1 (0.15 g, 0.32 mmol) in DCM (5 mL) was treated with TEA (0.054 mL, 0.37 mmol) and (4-methylphenyl)-carbonochloridic acid Compound 9a (0.06 g, 0.32 mmol). The reaction mixture was stirred for 4 hrs, then poured into water. The organic layer was separated, dried (Na₂SO₄), filtered and concentrated in vacuo to a residue which was purified by column chromatography (2:1:1 hexane:acetone:chloroform) to give Compound 4 (0.10 g) as a white solid. MS m/z 605 (M+H); M.P. 166-168° C.

### EXAMPLE 10

### (1-{3-[4-(2-isopropoxy-phenyl)-piperazin-1-ylmethyl]-benzoyl}-2,3-dihydro-1H-indol-6-yl)-carbamic acid p-tolyl ester (Cpd 12)

Using the procedure of Example 9, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 to provide Compound 12. MS m/z 605 (M+H⁺).

### EXAMPLE 11

### N-(1-{3-[4-(2-isopropoxy-phenyl)-piperazin-1-ylmethyl]-benzoyl}-2,3-dihydro-1H-indol-5-yl)-2,6-dimethoxy-benzamide (Cpd 26)

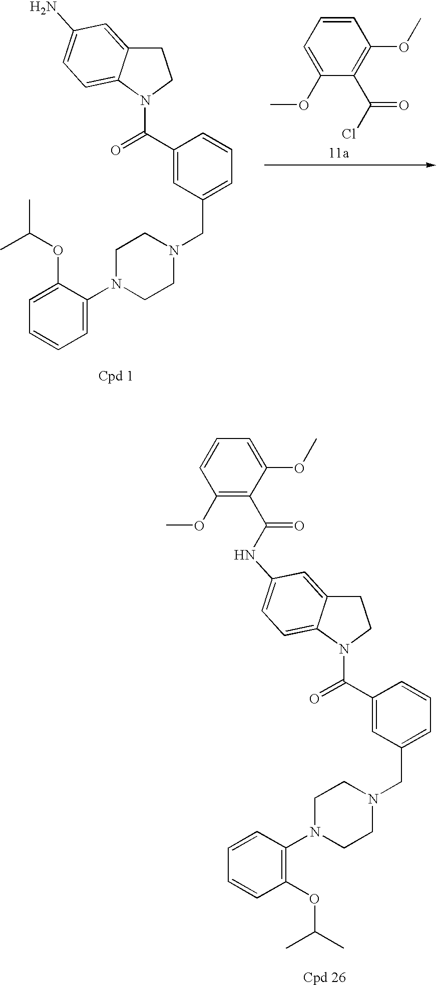

A solution of (5-amino-2,3-dihydro-indol-1-yl)-{3-[4-(2-isopropoxy-phenyl)-piperazin-1-ylmethyl]-phenyl}-methanone Compound 1 (0.15 g, 0.32 mmol) in DCM (5 mL) was treated with TEA (0.054 mL, 0.37 mmol) and 2,6-dimethoxy-benzoyl chloride Compound 11a (0.07 g, 0.32 mmol). The reaction mixture was stirred for 4 hrs and was then poured into water. The organic layer was separated, dried (Na₂SO₄), filtered and concentrated in vacuo to a residue which was purified by column chromatography (2:1:1 hexane:acetone:chloroform) to give Compound 26 (0.14 g) as a yellow solid. MS m/z 635 (M+H⁺); M.P. 229-232° C.

### EXAMPLE 12

### N-(1-{3-[4-(2-isopropoxy-phenyl)-piperazin-1-ylmethyl]-benzoyl}-2,3-dihydro-1H-indol-6-yl)-2,6-dimethoxy-benzamide (Cpd 28)

Using the procedure of Example 11, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1, to provide Compound 28. MS m/z 635 (M+H⁺).

Following the procedure of Examples 11 and 12, substituting the appropriate starting materials, reagents and solvents, the following compounds were prepared:

| Cpd | Name | MS |
|---|---|---|
| 40 | N-(1-{3-[4-(2-isopropoxy-phenyl)-piperazin-1-ylmethyl]- | 611 |
| | benzoyl}-2,3-dihydro-1H-indol-5-yl)-2,6-difluoro- | |
| | benzamide | |
| 42 | N-(1-{3-[4-(2-isopropoxy-phenyl)-piperazin-1-ylmethyl]- | 605 |
| | benzoyl}-2,3-dihydro-1H-indol-5-yl)-4-methoxy- | |
| | benzamide | |
| 48 | 4-fluoro-N-(1-{3-[4-(2-isopropoxy-phenyl)-piperazin-1- | 593 |
| | ylmethyl]-benzoyl}-2,3-dihydro-1H-indol-6-yl)-benzamide | |

### EXAMPLE 13

### N-(1-{3-[4-(2-isopropoxy-phenyl)-piperazin-1-ylmethyl]-benzoyl}-2,3-dihydro-1H-indol-5-yl)-sulfamide (Cpd 17)

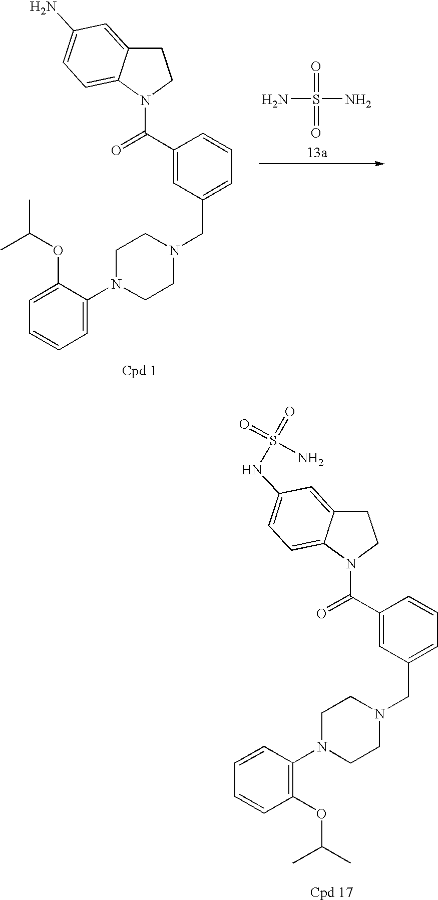

A solution of (5-amino-2,3-dihydro-indol-1-yl)-{3-[4-(2-isopropoxy-phenyl)-piperazin-1-ylmethyl]-phenyl}-methanone Compound 1 (0.30 g, 0.64 mmol) in dioxane (10 mL) was treated with sulfamide Compound 13a (0.30 g, 3.1 mmol). The reaction mixture was refluxed for 4 hrs and then was concentrated to a residue which was purified by column chromatography (30:30:1 acetone:chloroform:methanol) to give Compound 17 (0.075 g) as a brown solid. MS m/z 550 (M+H⁺).

### EXAMPLE 14

### N-(1-{3-[4-(2-isopropoxy-phenyl)-piperazin-1-ylmethyl]-benzoyl}-2,3-dihydro-1H-indol-6-yl)-sulfamide (Cpd 20)

Using the procedure of Example 13, (6-amino-2,3-dihydro-indol-1-yl)-{3-[4-(2-isopropoxy-phenyl)-piperazin-1-ylmethyl]-phenyl}-methanone Compound 5 was used in place of (5-amino-2,3-dihydro-indol-1-yl)-{3-[4-(2-isopropoxy-phenyl)-piperazin-1-ylmethyl]-phenyl}-methanone Compound 1 to provide Compound 20. MS m/z 550 (M+H⁺).

### EXAMPLE 15

### {3-[4-(2-isopropoxy-phenyl)-piperazin-1-ylmethyl]-phenyl}-[6-(1,2,3,4-tetrahydro-naphthalen-2-ylamino)-2,3-dihydro-indol-1-yl]-methanone (Cpd 16)

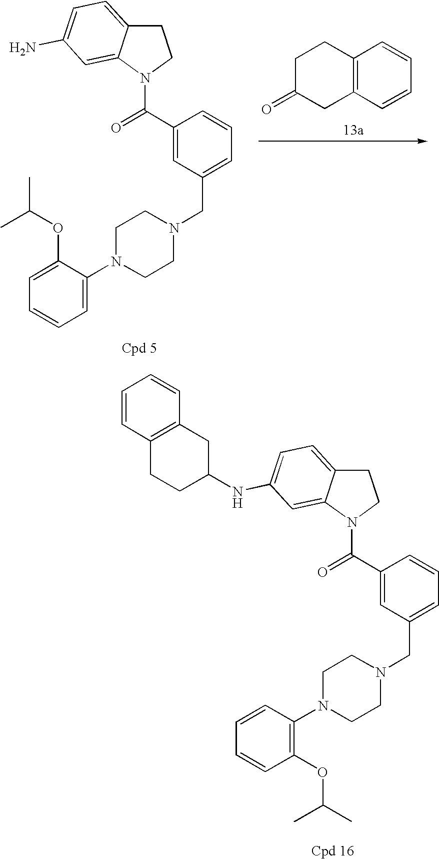

A solution of (6-amino-2,3-dihydro-indol-1-yl)-{3-[4-(2-isopropoxy-phenyl)-piperazin-1-ylmethyl]-phenyl}-methanone Compound 5 (0.15 g, 0.3 mmol) in DCM (4 mL) was treated with 2-tetralone (also known as 3,4-dihydro-1H-naphthalen-2-one) Compound 13a (0.046 g, 0.3 mmol), glacial acetic acid (0.02 mL) and sodium triacetoxyborohydride (0.15 g, 0.7 mmol) and stirred under an inert atmosphere for 24 hrs. The reaction mixture was treated with 1N NaOH (8 mL) and stirred for 60 min, then the layers were separated. The organic layer was dried (Na₂SO₄), filtered and concentrated in vacuo to give a residue which was purified by column chromatography (1:1:2 chloroform:acetone:hexanes) to give Compound 16 (0.049 g) as a syrup. MS m/z 601 (M+H).

### EXAMPLE 16

### [6-(4,7-dimethoxy-indan-2-ylamino)-2,3-dihydro-indol-1-yl]-{3-[4-(2-isopropoxy-phenyl)-piperazin-1-ylmethyl]-phenyl}-methanone (Cpd 29)

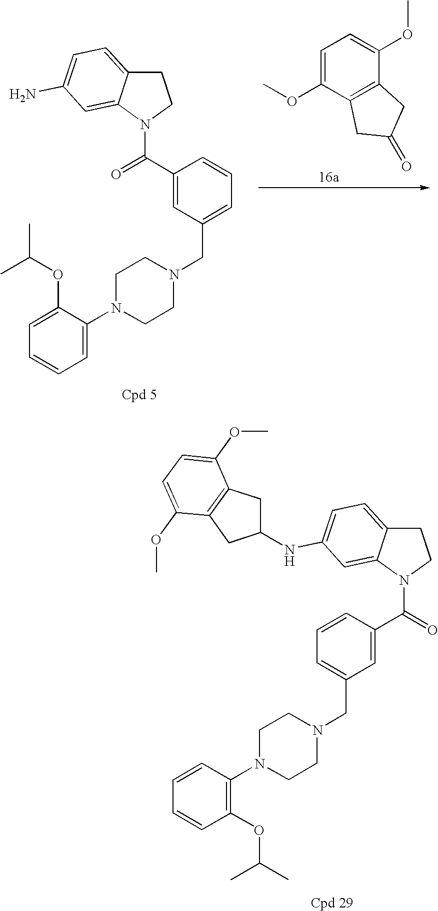

Using the procedure of Example 15, 4,7-dimethoxy-2-indanone Compound 16a was used in place of 3,4-dihydro-1H-naphthalen-2-one Compound 13a to provide Compound 29. MS m/z 647 (M+H⁺).

### EXAMPLE 17

### (5-amino-2,3-dihydro-indol-1-yl)-{4-[4-(2-methoxy-phenyl)-piperidin-1-ylmethyl]-phenyl}-methanone (Cpd 33) {4-[4-(2-methoxy-phenyl)-piperidin-1-ylmethyl]-phenyl}-(5-nitro-2,3-dihydro-indol-1-yl)-methanone (Cpd 38)

Using the procedure of Example 1,4-chloromethyl-benzoyl chloride Compound 17a was used in place of 3-chloromethyl-benzoyl chloride Compound 1b to provide (4-chloromethyl-phenyl)-(5-nitro-2,3-dihydro-indol-1-yl)-methanone Compound 17b.

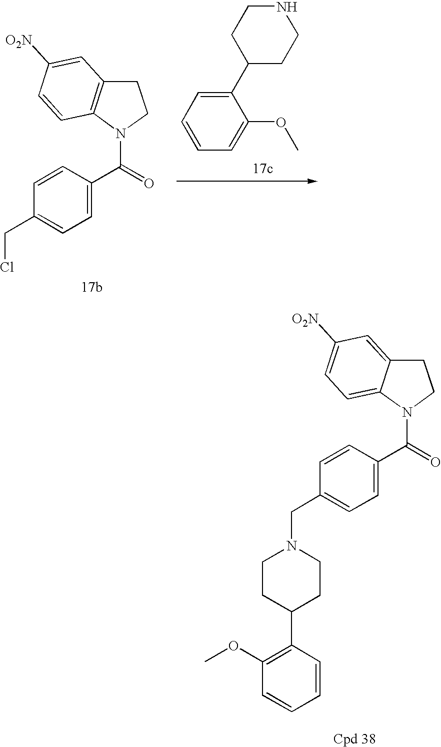

Using the procedure of Example 1, Compound 17b was used in place of (3-chloromethyl-phenyl)-(5-nitro-2,3-dihydro-indol-1-yl)-methanone Compound 1c and 4-(2-methoxy-phenyl)-piperidine Compound 17c was used in place of 1-(2-isopropoxy-phenyl)-piperazine monofumarate Compound 1d to provide Compound 38.

Using the procedure of Example 1, {4-[4-(2-methoxy-phenyl)-piperidin-1-ylmethyl]-phenyl}-(5-nitro-2,3-dihydro-indol-1-yl)-methanone Compound 38 was carried forward in place of {3-[4-(2-isopropoxy-phenyl)-piperazin-1-ylmethyl]-phenyl}-(5-nitro-2,3-dihydro-indol-1-yl)-methanone Compound 1e to provide Compound 33. MS m/z 442 (M+H⁺).

Following the procedure of Example 17, substituting the appropriate starting materials, reagents and solvents, the following compound(s) were prepared:

| Cpd | Name | MS |
|---|---|---|
| 50 | (2,3-dihydro-indol-1-yl)-{4-[4-(2-methoxy-phenyl)- | 427 |
| | piperidin-1-ylmethyl]-phenyl}-methanone | |

### EXAMPLE 18

### (6-amino-2,3-dihydro-indol-1-yl)-{4-[4-(2-methoxy-phenyl)-piperidin-1-ylmethyl]-phenyl}-methanone (Cpd 34) {4-[4-(2-methoxy-phenyl)-piperidin-1-ylmethyl]-phenyl}-(6-nitro-2,3-dihydro-indol-1-yl)-methanone (Cpd 35)

Using the procedure of Example 1,6-nitro-2,3-dihydro-1H-indole Compound 3a was used in place of 5-nitro-2,3-dihydro-1H-indole Compound 1a, 4-chloromethyl-benzoyl chloride Compound 17a was used in place of 3-chloromethyl-benzoyl chloride Compound 1b to provide (4-chloromethyl-phenyl)-(6-nitro-2,3-dihydro-indol-1-yl)-methanone Compound 18a.

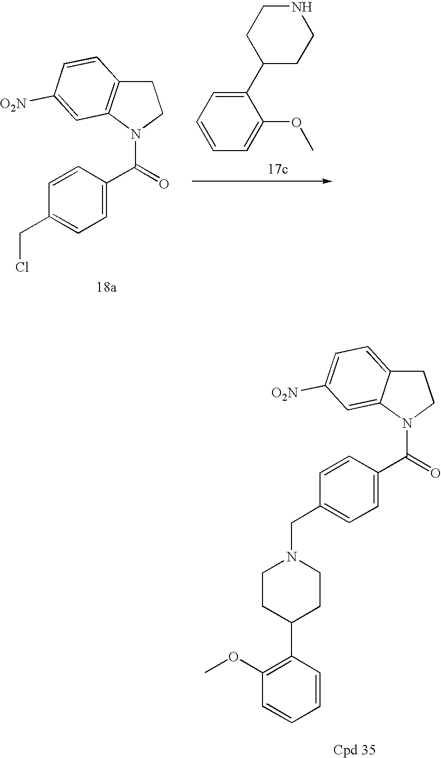

Using the procedure of Example 1, Compound 18a was used in place of (3-chloromethyl-phenyl)-(5-nitro-2,3-dihydro-indol-1-yl)-methanone Compound 1c and 4-(2-methoxy-phenyl)-piperidine Compound 17c was used in place of 1-(2-isopropoxy-phenyl)-piperazine monofumarate Compound 1d to provide Compound 35.

Using the procedure of Example 1, {4-[4-(2-methoxy-phenyl)-piperidin-1-ylmethyl]-phenyl}-(6-nitro-2,3-dihydro-indol-1-yl)-methanone Compound 35 was carried forward in place of {3-[4-(2-isopropoxy-phenyl)-piperazin-1-ylmethyl]-phenyl}-(5-nitro-2,3-dihydro-indol-1-yl)-methanone Compound 1e to provide Compound 34. MS m/z 442 (M+H⁺).

### EXAMPLE 19

### [5-(4-diethoxymethyl-benzylamino)-2,3-dihydro-indol-1-yl]-{4-[4-(2-methoxy-phenyl)-piperidin-1-ylmethyl]-phenyl}-methanone (Cpd 36)

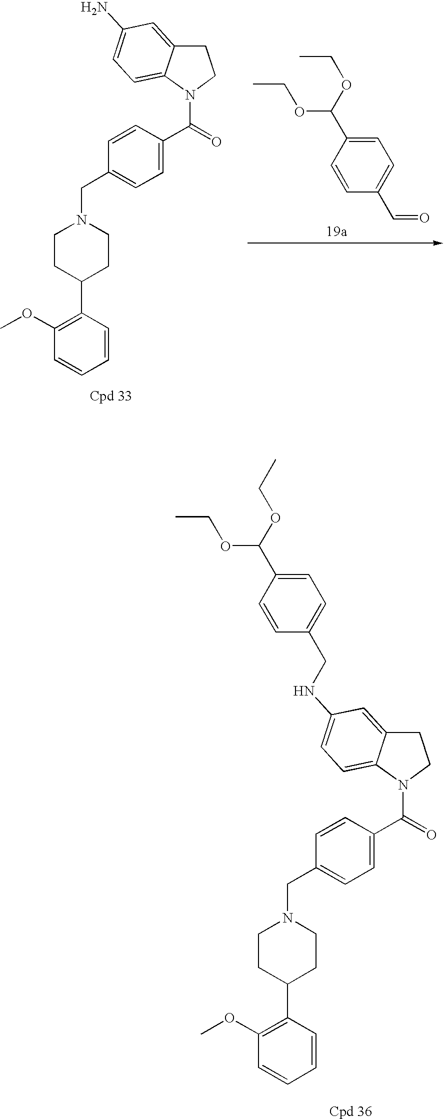

Using the procedure of Example 2, (5-amino-2,3-dihydro-indol-1-yl)-{4-[4-(2-methoxy-phenyl)-piperidin-1-ylmethyl]-phenyl}-methanone Compound 33 was used in place of (5-amino-2,3-dihydro-indol-1-yl)-{3-[4-(2-isopropoxy-phenyl)-piperazin-1-ylmethyl]-phenyl}-methanone Compound 1 and 4-diethoxymethyl-benzaldehyde Compound 19a was used in place of 4-trifluoromethyl-benzaldehyde Compound 2a to provide Compound 36 as a brown solid. MS m/z 643 (M+H⁺).

### EXAMPLE 20

### [6-(4-diethoxymethyl-benzylamino)-2,3-dihydro-indol-1-yl]-{4-[4-(2-methoxy-phenyl)-piperidin-1-ylmethyl]-phenyl}-methanone (Cpd 37)

Using the procedure of Example 2, (6-amino-2,3-dihydro-indol-1-yl)-{4-[4-(2-methoxy-phenyl)-piperidin-1-ylmethyl]-phenyl}-methanone Compound 34 was used in place of (5-amino-2,3-dihydro-indol-1-yl)-{3-[4-(2-isopropoxy-phenyl)-piperazin-1-ylmethyl]-phenyl}-methanone Compound 1 and 4-diethoxymethyl-benzaldehyde Compound 19a was used in place of 4-trifluoromethyl-benzaldehyde Compound 2a to provide Compound 37 as a white solid. MS m/z 643 (M+H⁺).

### EXAMPLE 21

### 2,6-dimethoxy-N-(1-{4-[4-(2-methoxy-phenyl)-piperidin-1-ylmethyl]-benzoyl}-2,3-dihydro-1H-indol-6-yl)-benzamide (Cpd 39)

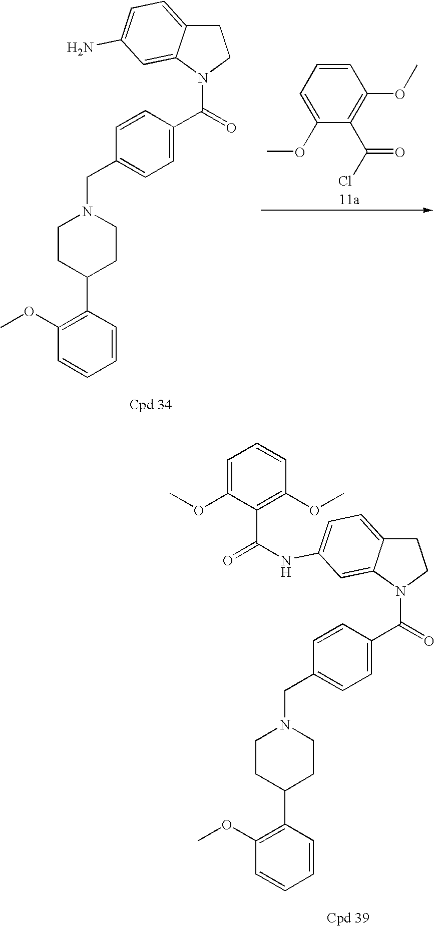

Using the procedure of Example 11, (6-amino-2,3-dihydro-indol-1-yl)-{4-[4-(2-methoxy-phenyl)-piperidin-1-ylmethyl]-phenyl}-methanone Compound 34 was used in place of (5-amino-2,3-dihydro-indol-1-yl)-{3-[4-(2-isopropoxy-phenyl)-piperazin-1-ylmethyl]-phenyl}-methanone Compound 1 to provide Compound 39 as a brown solid. MS m/z 606 (M+H⁺).

### EXAMPLE 22

### 2,6-difluoro-N-(1-{3-[4-(2-isopropoxy-phenyl)-piperidin 1-ylmethyl]-benzoyl}-2,3-dihydro-1H-indol-6-yl)-benzenesulfonamide (Cpd 58)

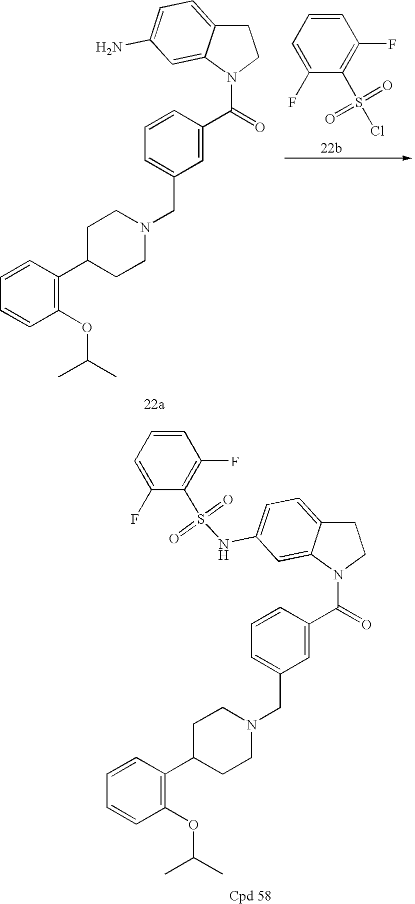

Using the procedure of Example 4, (6-amino-2,3-dihydro-indol-1-yl)-{3-[4-(2-isopropoxy-phenyl)-piperidin-1-ylmethyl]-phenyl}-methanone Compound 22a was used in place of (5-amino-2,3-dihydro-indol-1-yl)-{3-[4-(2-isopropoxy-phenyl)-piperazin-1-ylmethyl]-phenyl}-methanone Compound 1 and 2,6-difluoro-benzenesulfonyl chloride Compound 22b was used in place of naphthalene-2-sulfonyl chloride Compound 4a to provide Compound 58 as a cream-colored powder. MS m/z 646 (M+H⁺).

### EXAMPLE 23

### 2,6-difluoro-N-(1-{3-[4-(2-methoxy-phenyl)-piperidin-1-ylmethyl]-benzoyl}-2,3-dihydro-1H-indol-6-yl)-benzenesulfonamide (Cpd 61)

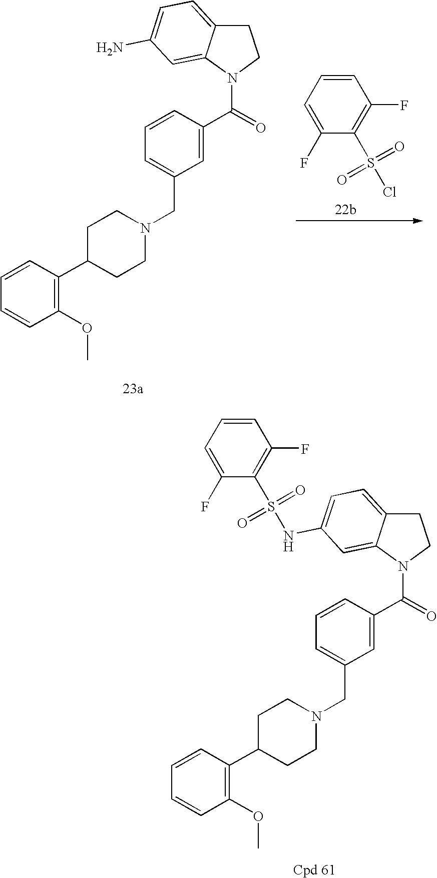

Using the procedure of Example 22, (6-amino-2,3-dihydro-indol-1-yl)-{3-[4-(2-methoxy-phenyl)-piperidin-1-ylmethyl]-phenyl}-methanone Compound 23a was used in place of (6-amino-2,3-dihydro-indol-1-yl)-{3-[4-(2-isopropoxy-phenyl)-piperidin-1-ylmethyl]-phenyl}-methanone Compound 22a to provide Compound 61 as a cream-colored powder. MS m/z 618 (M+H⁺).

### EXAMPLE 24

### 1-(2,6-difluoro-phenyl)-3-(1-{3-[4-(2-isopropoxy-phenyl)-piperidin-1-ylmethyl]-benzoyl}-2,3-dihydro-1H-indol-6-yl)-urea (Cpd 60)

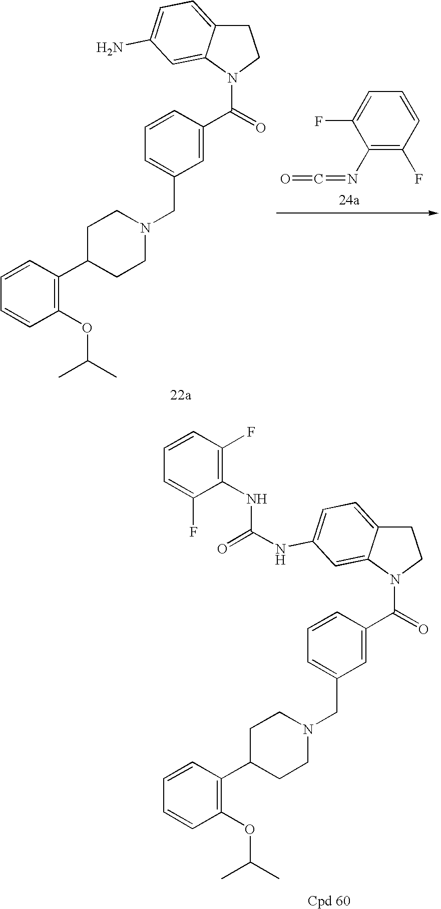

Using the procedure of Example 7, (6-amino-2,3-dihydro-indol-1-yl)-{3-[4-(2-isopropoxy-phenyl)-piperidin-1-ylmethyl]-phenyl}-methanone Compound 22a was used in place of (6-amino-2,3-dihydro-indol-1-yl)-{3-[4-(2-isopropoxy-phenyl)-piperazin-1-ylmethyl]-phenyl}-methanone Compound 5, and 1,3-difluoro-2-isocyanato-benzene Compound 24a was used in place of isocyanato-benzene Compound 6a to provide Compound 60 as a cream-colored powder. MS m/z 625 (M+H⁺).

### EXAMPLE 25

### (2,3-dihydro-indol-1-yl)-{4-[4-(2-isopropoxy-benzyl)-piperazin-1-ylmethyl]-phenyl}-methanone (Cpd 52)

Using the procedure of Example 1,2,3-dihydro-1H-indole Compound 25a was used in place of 5-nitro-2,3-dihydro-1H-indole Compound 1a and 4-chloromethyl-benzoyl chloride Compound 17a was used in place of 3-chloromethyl-benzoyl chloride Compound 1b to provide (4-chloromethyl-phenyl)-(2,3-dihydro-indol-1-yl)-methanone Compound 25b.

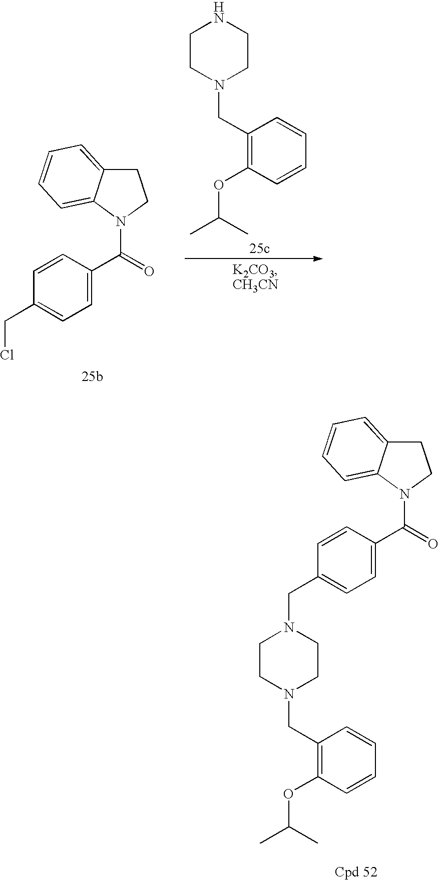

Compound 25b was used in place of (3-chloromethyl-phenyl)-(5-nitro-2,3-dihydro-indol-1-yl)-methanone Compound 1c and 1-(2-isopropoxy-benzyl)-piperazine monofumarate Compound 25c was used in place of 1-(2-isopropoxy-phenyl)-piperazine monofumarate Compound 1d to provide Compound 52. MS m/z 470 (M+H⁺).

Following the procedure of Example 25, substituting the appropriate starting materials, reagents and solvents, the following compound(s) were prepared:

| Cpd | Name | MS |
|---|---|---|
| 53 | (2,3-dihydro-indol-1-yl)-{4-[4-(2-methoxy-benzyl)- | 442 |
| | piperazin-1-ylmethyl]-phenyl}-methanone | |
| 55 | (2,3-dihydro-indol-1-yl)-(4-{4-[2-(2,2,2-trifluoro-ethoxy)- | 496 |
| | phenyl]-piperazin-1-ylmethyl}-phenyl)-methanone | |
| 62 | (2,3-dihydro-indol-1-yl)-{4-[4-(2-hydroxy-benzyl)- | 428 |
| | piperazin-1-ylmethyl]-phenyl}-methanone | |

### EXAMPLE 26

(2,3-dihydro-indol-1-yl)-{3-[4-(2-isopropoxy-phenyl)-piperidin-1-ylmethyl]-phenyl}-methanone (Cpd 51)

Using the procedure of Example 1,2,3-dihydro-1H-indole Compound 25a was used in place of 5-nitro-2,3-dihydro-1H-indole Compound 1a to provide (3-chloromethyl-phenyl)-(2,3-dihydro-indol-1-yl)-methanone Compound 26a.

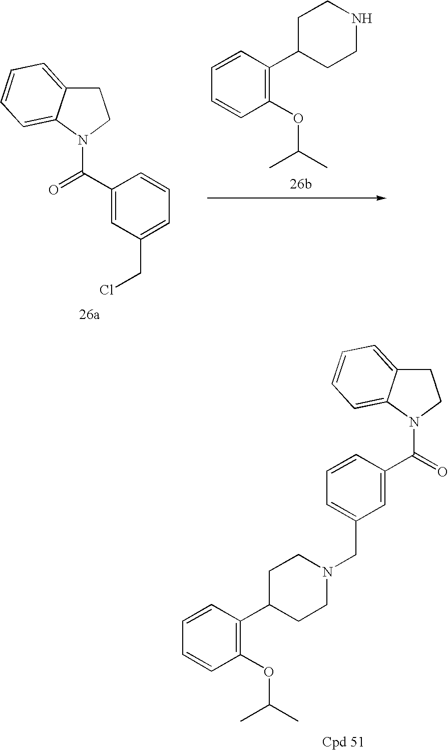

Compound 26a was used in place of (3-chloromethyl-phenyl)-(5-nitro-2,3-dihydro-indol-1-yl)-methanone Compound 1c and 4-(2-isopropoxy-phenyl)-piperidine Compound 26b was used in place of 1-(2-isopropoxy-phenyl)-piperazine monofumarate Compound 1d to provide Compound 51. MS m/z 455 (M+H⁺).

Following the procedure of Example 26, substituting the appropriate starting materials, reagents and solvents, the following compounds were prepared:

| Cpd | Name | MS |
|---|---|---|
| 57 | [6-(2-fluoro-benzylamino)-2,3-dihydro-indol-1-yl]-{3- | 578 |
| | [4-(2-isopropoxy-phenyl)-piperidin-1-ylmethyl]-phenyl}- | |
| | methanone | |
| 59 | [6-(2-fluoro-benzylamino)-2,3-dihydro-indol-1-yl]-{3-[4- | 549 |
| | (2-methoxy-phenyl)-piperidin-1-ylmethyl]-phenyl}- | |
| | methanone | |

### BIOLOGICAL EXAMPLES

α1-Adrenergic Receptor Binding Assay

Preparation of COS Cell Membranes

Membranes were prepared from COS-7 cells (African Green monkey kidney SV40-transformed cells) that had been transfected with one of the three α₁-AR subtypes by the following method:

COS cells from ten 100 mm tissue culture plates were scraped into a 5 mL volume of TE (a mixture of 50 mM tris(hydroxymethyl)aminomethane hydrochloride (Tris-HCl) and 5 mM ethylenediaminetetraacetic acid (EDTA) at pH 7.4). The cell suspension was disrupted with a Brinkman Polytron (at a setting of 8) for 10 sec. The disrupted cells were centrifuged at 1000×g for 10 min at 4° C. Supernatants were centrifuged at 34,500×g for 20 min at 4° C. The membrane pellets were suspended in a 2 mL volume of TNE (a mixture of 50 mM Tris-HCl, 5 mM EDTA and 150 mM NaCl at pH7.4). An aliquot of the membrane suspension was stored at −70° C. until use. The protein concentration was determined using a BioRad “DC” protein assay kit following membrane solubilization with Triton X-100.

Radio-Ligand Binding Assay

Triplicate determinations of radio-ligand binding in the presence of increasing concentrations of testing compound were made. The reagents were added to 96-well polypropylene plate wells. Each assay well contained 140 μL TNE, 25 μL ¹²⁵I-2-(β-4-hydroxyplenyl)ethylaminomethyltetralone (¹²⁵I-HEAT) (specific activity 2200 Ci/mmol, Dupont-New England Nuclear, 50 pM final), 10 μL testing compound dissolved in dimethyl sulfoxide (DMSO) (1 pM to 10 μM in half-log increments, final), and 25 μL appropriate α₁-AR membrane subtype suspension in TNE (0.5 ng/μL for the α₁ₐ and α_{1b} subtypes and 13 ng/μL for the α_{1d} subtype). The plate was incubated at rt for 1 hr. The contents of the wells were filtered through a glass filter (type C) (GF/C) membrane Unifilter plate (Packard Instruments) using the Packard Filtermate cell harvester. The filter plates were dried in a vacuum oven for 30 min at 40° C. 25 μL Microscint 20 liquid scintillation fluid (Packard Instuments) was added to each well. The radioactive content was analyzed in the TopCount microplate scintillation counter (Packard Instruments).

Data Analysis

The Kᵢ values (in nM) shown in Table 1 were determined using GraphPad Prism software. K_{d} values used in the Kᵢ calculation for the α₁-AR subtypes for ¹²⁵I-HEAT were 81.5 nM for the α₁ₐ-AR, 79 nM for the α_{1b}-AR and 50 nM for the α_{1d}-AR.

TABLE 1
Receptor Binding, Kᵢ (nM)

| Cpd | α1a-AR | α1b-AR | α1d-AR |
|---|---|---|---|
| 1 | 1.5 | 301 | 8.6 |
| 2 | 1.1 | 321 | 9.7 |
| 3 | 65 | 3452 | 98 |
| 4 | 2.4 | 96 | 20 |
| 5 | 1.9 | 143 | 2 |
| 6 | 2 | 856 | 2.4 |
| 7 | 11 | 684 | 16 |
| 8 | 14 | 389 | 15 |
| 9 | 26 | 2054 | 17 |
| 10 | 836 | 5000 | 250 |
| 11 | 5 | 6259 | 7 |
| 12 | 3.3 | 495 | 5.7 |
| 13 | 33 | 305 | 4/3.11 |
| 14 | 22 | 5000 | 5000/4 |
| 15 | 1 | 91 | 700/5000 |
| 16 | 5.5 | 282 | 3.4 |
| 17 | 14 | 812 | 7.6 |
| 18 | 0.04 | 43 | 4 |
| 19 | 26 | 383 | 2.3 |
| 20 | 4.7/8.1 | 494/394 | 1.9/2.6 |
| 21 | 73 | 1395 | 12.5 |
| 22 | 39 | 251 | 5 |
| 23 | 195 | 5000 | 317 |
| 24 | 48 | 1472 | 15 |
| 25 | 67 | 1303 | 18 |
| 26 | 34 | 200 | 2 |
| 27 | 16.5 | 379 | 11 |
| 28 | 17 | 796 | 6.8 |
| 29 | 20.8 | 847 | 10.2/750 |
| 30 | 26/30.5 | 277.5/192.8 | 4.5/42 |
| 31 | 171 | 331 | 11.6/88 |
| 32 | 2.7 | 610 | 9.4 |
| 33 | 48.8 | 658.1 | 49.1 |
| 34 | 34.9 | 473.1 | 49.6 |
| 35 | 5.3 | 200.7 | 8.5 |
| 36 | 39.2 | 1012 | 40.2 |
| 37 | 75.6 | 909.4 | 98 |
| 38 | 124 | 461 | 44.2 |
| 39 | 74 | 595 | 41 |
| 40 | 31 | 208 | 1.5 |
| 41 | 31 | 208 | 1.5 |
| 42 | 3.8 | 447 | 5.8 |
| 43 | 7.8/3.4 | 204/185 | 2.8/3.2 |
| 44 | 1.3 | 5000 | 4.2 |
| 45 | 2.6 | 888 | 4 |
| 46 | 5.9 | 242 | 5.6 |
| 47 | 2.3 | 230 | 2.6 |
| 48 | 74 | 889 | 6 |
| 49 | 14 | 351 | 13 |
| 50 | 7.8 | 458 | 32 |
| 51 | 0.8 | 106 | 1.6 |
| 52 | 122 | 1733 | 159 |
| 53 | 82 | 104 | 96 |
| 54 | 67.5 | 5000 | 5000 |
| 55 | 25.5 | 310 | 43 |
| 56 | 1197 | 655 | 1907 |
| 57 | 1.8 | 237 | 1.4 |
| 58 | 0.8/0.5 | 234/254.7 | 1.9/1 |
| 59 | 8.3 | 100.5 | 1.1 |
| 60 | 0.4 | 120.9 | 0.4 |
| 61 | 15.5 | 152.2 | 2.1 |
| 62 | | | |
| 63 | 0.1/2.1 | 10.6/117.3 | 0.5/1.9 |
| 64 | 105 | 146 | 101 |
| 65 | 65 | 909 | 42 |
| 66 | 1.3 | 82.8 | 0.3 |
| 67 | 2.1/12 | 1142/1260 | 14.7/22 |
| 68 | 13 | 288 | 13 |
| 69 | 0.36/15 | 2.8/170 | 0.25/4.3 |
| 70 | 107 | 1760 | 41 |
| 71 | 14/17.5 | 716/720 | 16/21 |
| 72 | 15 | 154 | 46 |
| 73 | 31 | 208 | 1.5 |

In Vivo Models

The ability of a test compound to relax prostatic smooth muscle tissue in vivo may be evaluated using the prostatic intraurethral pressure (IUP) and blood pressure (MAP) in the anesthetized canine model. Alternatively, the ability of a test compound to relax prostate smooth muscle tissue in vivo may be evaluated by evaluating the prostatic intraurethral pressure (IUP) and blood pressure (MAP) in the conscious canine model.

It is to be understood that the preceding description teaches the principles of the present invention, with examples thereof, which have emphasized certain aspects. It will also be understood that the practice of the invention encompasses all of the usual variations, adaptations and modifications as come within the scope of the following claims and their equivalents. However, numerous other equivalents not specifically elaborated on or discussed may nevertheless fall within the spirit and scope of the present invention and claims and are intended to be included.

Throughout this application, various publications are cited. The disclosure of all publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats.

What is claimed is:

1. A compound of Formula (I) 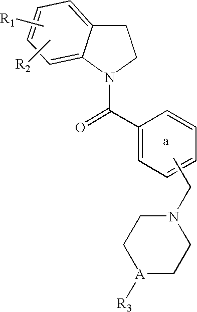
or a pharmaceutically acceptable form thereof, wherein
“a” represents a point of attachment selected from the 3 or 4 position on the phenyl ring relative to the point of attachment of the methanone group for the compound of Formula (I),
“A” is a ring atom selected from CH or N,
R₁ is one substituent selected from the group consisting of hydrogen, —N—, halogen and nitro, wherein —N— is substituted with two substituents independently selected from the group consisting of hydrogen, C₁₋₈alkyl, C₁₋₈alkyl(C₁₋₈alkoxy), C(O)(R^{A}), C(O)O(R^{A}), C(O)NH₂, C(O)NH(C₁₋₈alkyl), C(O)N(C₁₋₈alkyl)₂, C(O)NH(R^{A}), C(O)N(R^{A})₂, C(O)NH(C₁₋₈alkyl-R^{A}), C(O)N(C₁₋₈alkyl-R^{A})₂, C(S)NH(R^{A}), C(S)N(R^{A})₂, C(S)NH(C₁₋₈alkyl-R^{A}), C(S)N(C₁₋₈alkyl-R^{A})₂, SO₂(C₁₋₈alkyl), SO₂(R^{A}), SO₂NH₂, SO₂NH(C₁₋₈alkyl), SO₂N(C₁₋₈alkyl)₂, SO₂NH(R^{A}), C₁₋₈alkyl(R^{A}) and R^{A},
R₂ is one substituent selected from the group consisting of hydrogen, —SO₂— and R^{A}, wherein —SO₂— is substituted with C₁₋₈alkyl, NH₂, NH(C₁₋₈alkyl) or N(C₁₋₈alkyl)₂,
R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), NH₂, NH(C₁₋₈alkyl), N(C₁₋₈alkyl)₂, halogen, hydroxy, C(O)NH₂, C(O)NH(C₁₋₈alkyl), C(O)N(C₁₋₈alkyl)₂, NHC(O)H and NHC(O)(C₁₋₈alkyl),
R₃ is selected from the group consisting of C₁₋₈alkyl(R^{B}), —C(O)(C₁₋₈alkoxy) and R^{B}, and
R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), C₁₋₈alkoxy(hydroxy), cyano, halogen and hydroxy,
with the proviso that (2,3-dihydro-indol-1-yl)-{4-[4-(2-isopropoxy-phenyl)-piperazine-1-ylmethyl]-phenyl}-methanone is not included as a compound of Formula (I).

2. A compound according to claim 1, wherein R₁ is one substituent selected from the group consisting of hydrogen, —N—, halogen and nitro, wherein —N— is substituted with two substituents independently selected from the group consisting of hydrogen, C(O)(R^{A}), C(O)O(R^{A}), C(O)NH₂, C(O)NH(R^{A}), C(O)N(R^{A})₂, C(O)NH(C₁₋₈alkyl-R^{A}), C(O)N(C₁₋₈alkyl-R^{A})₂, C(S)NH(R^{A}), C(S)N(R^{A})₂, C(S)NH(C₁₋₈alkyl-R^{A}), C(S)N(C₁₋₈alkyl-R^{A})₂, SO₂(C₁₋₈alkyl), SO₂(R^{A}), SO₂NH₂, SO₂NH(R^{A}), C₁₋₈alkyl(R^{A}) and R^{A}.

3. A compound according to claim 1, wherein R₁ is one substituent selected from the group consisting of hydrogen, —N—, halogen and nitro, wherein —N— is substituted with two substituents independently selected from the group consisting of hydrogen, C(O)(R^{A}), C(O)O(R^{A}), C(O)NH₂, C(O)NH(R^{A}), C(S)NH(R^{A}), SO₂(C₁₋₈alkyl), SO₂(R^{A}), SO₂NH₂, C₁₋₈alkyl(R^{A}) and R^{A}.

4. A compound according to claim 1, wherein R₂ is one substituent selected from the group consisting of hydrogen, —SO₂— and R^{A}, wherein —SO₂— is substituted with C₁₋₈alkyl or N(C₁₋₈alkyl)₂.

5. A compound according to claim 1, wherein R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, NH₂, NH(C₁₋₈alkyl), N(C₁₋₈alkyl)₂, halogen, hydroxy and NHC(O)(C₁₋₈alkyl).

6. A compound according to claim 1, wherein R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, N(C₁₋₈alkyl)₂, halogen and NHC(O)(C₁₋₈alkyl).

7. A compound according to claim 1, wherein R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(halogen)₁₋₃, C₁₋₈alkoxy(halogen)₁₋₃, cyano, halogen and hydroxy.

8. A compound according to claim 1, wherein R^{B} is aryl optionally substituted with one to three substituents independently selected from the group consisting of C₁₋₁₀alkyl, C₁₋₈alkoxy, C₁₋₈alkyl(halogen)₁₋₃, C₁₋₈alkoxy(halogen)₁₋₃, cyano, halogen and hydroxy.

9. A compound according to claim 1, wherein R^{B} is aryl optionally substituted with one to three substituents independently selected from the group consisting of C₁₋₈alkoxy, C₁₋₈alkoxy(halogen)₁₋₃, cyano and hydroxy.

10. A compound of formula (Ia): 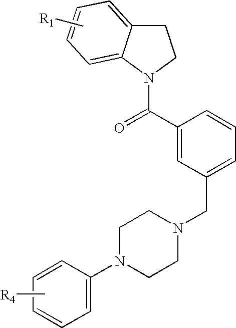
or a pharmaceutically acceptable form thereof, wherein
R₁ is selected from the group consisting of hydrogen, 5-NH₂, 5-NHSO₂-naphthalen-2-yl, 5-NHSO₂-5-N(CH₃)₂-naphthalen-1-yl, 5-NHC(O)O-4-CH₃-phenyl, 6-NH₂, 6-NHCH₂-2,6-F₂-phenyl, 6-NHCH₂-2,6-(OCH₃)₂-phenyl, 5-NHCH₂-4-CF₃-phenyl, 5-NHCH₂-2,6-F₂-phenyl, 6-NHCH₂-4-(CH₂)₇CH₃-phenyl, 6-NHCH₂-4-CH(OCH₂CH₃)₂-phenyl, 6-NHC(O)O-4-CH₃-phenyl, 6-NHC(S)NH-phenyl, 5-NHC(O)NH-2,4-Cl₂-phenyl, 6-NHSO₂-4-NHC(O)CH₃-phenyl, 6-NH-1,2,3,4-tetrahydro-naphthalen-2-yl, 5-NHSO₂NH₂, 6-NHSO₂-5-Cl-3-CH₃-benzo[b]thien-2-yl, 5-NHSO₂-4-NHC(O)CH₃-phenyl, 6-NHSO₂NH₂, 5-NHC(O)NH-phenyl, 6-NHC(O)NH-phenyl, 5-NHCH₂-4-(CH₂)₇CH₃-phenyl, 5-NHCH₂-4-CH(OCH₂CH₃)₂-phenyl, 5-NHCH₂-2,6-(OCH₃)₂-phenyl, 5-NHC(O)-2,6-(OCH₃)₂-phenyl, 5-NHCH₂-benzo[1,3]dioxol-5-yl, 6-NHC(O)-2,6-(OCH₃)₂-phenyl, 6-NH-4,7-(OCH₃)₂-indan-2-yl, 6-NHSO₂-5-N(CH₃)₂-naphthalen-1-yl, 6-NHC(O)-2,6-F₂-phenyl, 6-NHCH₂-benzo[1,3]dioxol-5-yl, 6-NHC(O)-4-OCH₃-phenyl, 6-N[C(O)NH-2,6-F₂-phenyl]₂, 6-NHCH₂-2-F-phenyl, 6-NHSO₂-2,6-F₂-phenyl, 6-NHSO₂-4-CH₃-phenyl, 6-NHSO₂CH₂CH₃, 6-NHC(O)-4-F-phenyl, 6-NHC(O)NH₂, 6-NHC(O)NH-2,6-F₂-phenyl, 5-Br, 5-Cl and 5-F; and
R₄ is selected from the group consisting of hydrogen, 2-OCH(CH₃)₂, 2-OCH₃, 2-CN, 2-OCH₂CF₃, 2-OCH₂CH(CH₃)₂ and 2-OCH₂CH₃.

11. A compound of Formula (Ib): 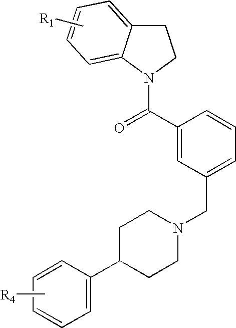
or a pharmaceutically acceptable form thereof, wherein
R₁ is selected from the group consisting of hydrogen, 6-NHCH₂-2-F-phenyl, 6-NHSO₂-2,6-F₂-phenyl, 6-NHCH₂-2-F-phenyl, 6-NHC(O)NH-2,6-F₂-phenyl, and 6-NHSO₂-2,6-F₂-phenyl; and
R₄ is selected from the group consisting of 2-OCH(CH₃)₂ and 2-OCH₃.

12. A compound of Formula (Ic): 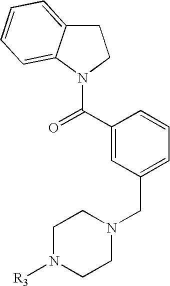
or a pharmaceutically acceptable form thereof, wherein
R₃ is selected from the group consisting of C(O)OC(CH₃)₃, CH₂-2-OCH(CH₃)₂-phenyl and CH₂-2-OCH₃-phenyl.

13. A compound of Formula (Id): 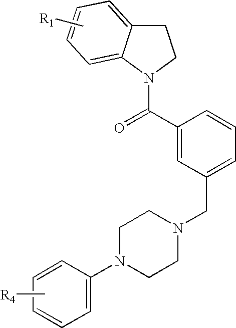
or a pharmaceutically acceptable form thereof, wherein
R₂ is selected from the group consisting of 5-SO₂N(Me)₂, 5-SO₂Me and 5-Ph; and
R₄ is 2-OCH₂CH(CH₃)₂.

14. A compound of Formula (Ie): 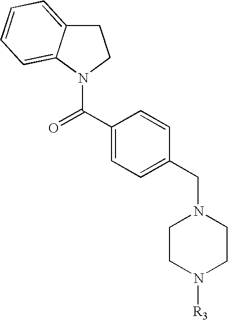
or a pharmaceutically acceptable form thereof, wherein
R₃ is selected from the group consisting of CH₂-2-OCH(CH₃)₂-phenyl, CH₂-2-OCH₃-phenyl, 2-OCH₂CF₃-phenyl, and CH₂-2-OH-phenyl.

15. A compound of Formula (If): 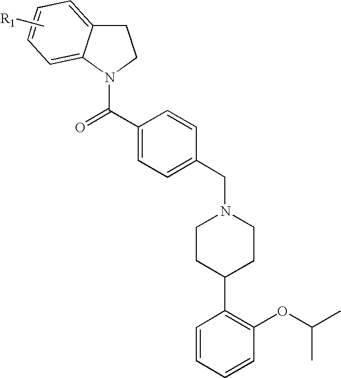
or a pharmaceutically acceptable form thereof, wherein
R₁ is selected from the group consisting of 5-NH₂, 6-NH₂, 6-NO₂, 5-NHCH₂-4-CH(OCH₂CH₃)₂-phenyl, 6-NHCH2-4-CH(OCH₂CH₃)₂-phenyl, 5-NO₂, 6-NHC(O)-2,6-(OCH₃)₂-phenyl and hydrogen.

16. The compound of any of claim 1 to 15, wherein the compound is an α₁ₐ/α_{1d} adrenoreceptor modulator.

17. The compound of claim 16, wherein the compound is a prodrug form thereof.

18. The compound of any of claim 1 to 17, wherein the compound is an isolated form thereof.

19. An α₁ₐ/α_{1d} adrenoreceptor modulator characterized in that it is a compound as claimed in claim 1.

20. An α₁ₐ/α_{1d} adrenoreceptor antagonist characterized in that it is a compound as claimed in claim 1.

21. The compound of claim 17, wherein the compound is a metabolite form thereof.

22. The compound of any of claim 1 to 18, wherein the compound is labeled with a ligand for use as a marker, and wherein the ligand is a radioligand selected from deuterium or tritium.

23. A pharmaceutical composition comprising an effective amount of the compound of any of claim 1 to 18 and a pharmaceutically acceptable carrier.

24. The pharmaceutical composition of claim 23, wherein the effective amount of the compound is in a range of from about 0.001 mg/kg to about 300 mg/kg of body weight per day.

25. A process for preparing a pharmaceutical composition comprising the step of intimately mixing the compound of any of claim 1 to 18 and a pharmaceutically acceptable carrier.

26. A medicament comprising an effective amount of the compound of any of claim 1 to 18.

27. The medicament of claim 26, wherein the effective amount of the compound is in a range of from about 0.001 mg/kg to about 300 mg/kg of body weight per day.

28. A medicine comprising an effective amount of the compound of any of claim 1 to 18.

29. The medicine of claim 28, wherein the effective amount of the compound is in a range of from about 0.001 mg/kg to about 300 mg/kg of body weight per day.

30. Use of the compound of any of claim 1 to 18 as an α₁ₐ/α_{1d} adrenoreceptor antagonist comprising contacting one or both of the α₁ₐ or α_{1d} adrenoreceptors with the compound.

31. The use of claim 30, wherein the use further comprises use of the compound in a pharmaceutical composition, medicine or medicament for the treatment of an α₁ₐ/α_{1d} adrenoreceptor mediated disease.

32. Use of the compound of any of claim 1 to 18 for the manufacture of a medicament for treating an α₁ₐ adrenoreceptor, an α_{1d} adrenoreceptor or a dual α₁ₐ/α_{1d} adrenoreceptor mediated disease.

33. A method of treating an α₁ₐ adrenoreceptor, an α_{1d} adrenoreceptor or a dual α₁ₐ/α_{1d} adrenoreceptor mediated disease, comprising administering to a patient in need of such treatment an effective amount of a compound of formula (II): 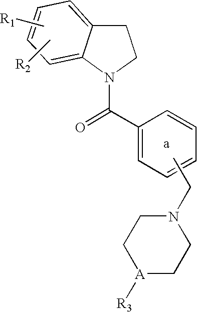
or a pharmaceutically acceptable form thereof, wherein
“a” represents a point of attachment selected from the 3 or 4 position on the phenyl ring relative to the point of attachment of the methanone group for the compound of Formula (I),
“A” is a ring atom selected from CH or N,
R₁ is one substituent selected from the group consisting of hydrogen, —N—, halogen and nitro, wherein —N— is substituted with two substituents independently selected from the group consisting of hydrogen, C₁₋₈alkyl, C₁₋₈alkyl(C₁₋₈alkoxy), C(O)(R^{A}), C(O)O(R^{A}), C(O)NH₂, C(O)NH(C₁₋₈alkyl), C(O)N(C₁₋₈alkyl)₂, C(O)NH(R^{A}), C(O)N(R^{A})₂, C(O)NH(C₁₋₈alkyl-R^{A}), C(O)N(C₁₋₈alkyl-R^{A})₂, C(S)NH(R^{A}), C(S)N(R^{A})₂, C(S)NH(C₁₋₈alkyl-R C(S)N(C₁₋₈alkyl-R^{A})₂, SO₂(C₁₋₈alkyl), SO₂(R^{A}), SO₂NH₂, SO₂NH(C₁₋₈alkyl), SO₂N(C₁₋₈alkyl)₂, SO₂NH(R^{A}), C₁₋₈alkyl(R^{A}) and R^{A},
R₂ is one substituent selected from the group consisting of hydrogen, —SO₂— and R^{A}, wherein —SO₂— is substituted with C₁₋₈alkyl, NH₂, NH(C₁₋₈alkyl) or N(C₁₋₈alkyl)₂,
R^{A} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), NH₂, NH(C₁₋₈alkyl), N(C₁₋₈alkyl)₂, halogen, hydroxy, C(O)NH₂, C(O)NH(C₁₋₈alkyl), C(O)N(C₁₋₈alkyl)₂, NHC(O)H and NHC(O)(C₁₋₈alkyl),
R₃ is selected from the group consisting of C₁₋₈alkyl(R^{B}), —C(O)(C₁₋₈alkoxy) and R^{B}, and
R^{B} is selected from the group consisting of C₃₋₁₂cycloalkyl, heterocyclyl, aryl and heteroaryl, wherein each is optionally substituted with one to three substituents independently selected from the group consisting of C₁₋₁₀alkyl, C₁₋₈alkoxy, C₁₋₈alkyl(C₁₋₈alkoxy)₁₋₂, C₁₋₈alkyl(halogen)₁₋₃, C₁₋₈alkoxy(halogen)₁₋₃, C₁₋₈alkyl(hydroxy), C₁₋₈alkoxy(hydroxy), cyano, halogen and hydroxyl.

34. The method of claim 33, wherein the compound is an α₁ₐ adrenoreceptor, an α_{1d} adrenoreceptor or a dual α₁ₐ/α_{1d} modulator.

35. The method of claim 34, wherein the compound is a prodrug form thereof.

36. The method of claim 34, wherein the compound is an isolated form thereof.

37. The method of claim 35, wherein the compound is a metabolite form thereof.

38. The method of claim 33, wherein the effective amount is in a range of from about 0.001 mg/kg to about 300 mg/kg of body weight per day.

39. The method of claim 33, further comprising the use of an effective amount of the compound or a pharmaceutical composition, medicine or medicament thereof for the treatment of an α₁ₐ adrenoreceptor, an α_{1d} adrenoreceptor or a dual α₁ₐ/α_{1d} mediated disease.

40. The method of claim 39, wherein the pharmaceutical composition comprises an effective amount of the compound and a pharmaceutically acceptable carrier.

41. The method of claim 40, wherein the effective amount is in a range of from about 0.001 mg/kg to about 300 mg/kg of body weight per day.

42. The method of claim 40, wherein the pharmaceutical composition is prepared by the step of intimately mixing the compound and a pharmaceutically acceptable carrier.

43. The method of claim 39, wherein the medicament comprises an effective amount of the compound.

44. The method of claim 43, wherein the effective amount is in a range of from about 0.001 mg/kg to about 300 mg/kg of body weight per day.

45. The method of claim 39, wherein the medicine comprises an effective amount of the compound.

46. The method of claim 45, wherein the effective amount of the compound is in a range of from about 0.001 mg/kg to about 300 mg/kg of body weight per day.

47. The method of claim 39, further comprising contacting one or both of the α₁ₐ or α_{1d} adrenoreceptors with the compound.

48. Use of the compound of claim 33 in the manufacture of a medicament for treating, preventing or ameliorating a kinase mediated disease, disorder or condition.

49. The method of claim 33, wherein the α₁ₐ adrenoreceptor, α_{1d} adrenoreceptor or dual α₁ₐ/α_{1d} adrenoreceptor mediated disease is LUTS.

49. The method of claim 33, wherein the α₁ₐ adrenoreceptor, α_{1d} adrenoreceptor or dual α₁ₐ/α₁ adrenoreceptor mediated disease is BPH.

50. Use of the compound of claim 33 for the manufacture of a medicament for treating an ala adrenoreceptor, an α_{1d} adrenoreceptor or a dual α₁ₐ/α_{1d} adrenoreceptor mediated disease.

51. A process for preparing the compound of any of claim 1 to 18 or claim 33, comprising the steps of:
a) reacting an intermediate of formula A1 with an intermediate of formula A2 resulting in an intermediate of formula A3: 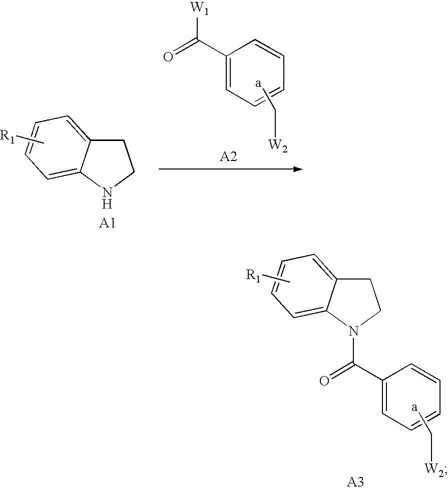
b) reacting an intermediate of formula A3 with an intermediate of formula A4, resulting in a compound of formula A5: 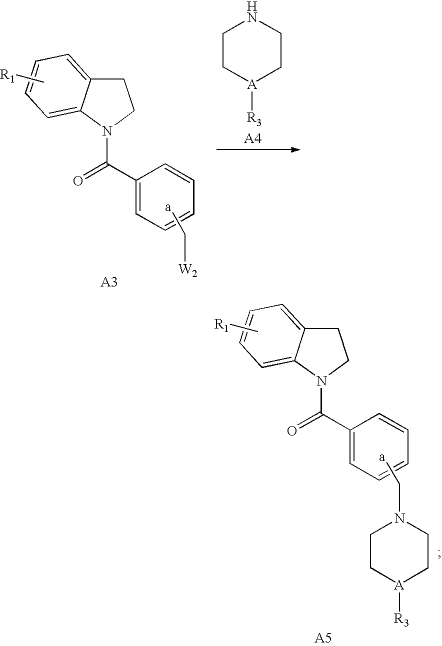
c) converting compounds of formula A5 into compounds of general formula (I) by art-known functional group transformations and optionally preparing a pharmaceutically acceptable salt thereof.
